(19) United States
(12) Patent Application Publication
(10) Pub. No.: US 2026/0286312 A1
(43) Pub. Date: Sep. 24, 2026

(54) METHODS AND COMPOSITIONS FOR OVARIAN ORGANOID CULTURE

(71) Applicant: Conception Biosciences, Inc., Berkeley, CA (US)

(72) Inventors: Tisha E. BOHR, Berkeley, CA (US); Alyssa J. MILLER, Berkeley, CA (US); Pablo Ignacio HURTADO GONZALEZ, Berkeley, CA (US); Karmen Bianka SERES, Berkeley, CA (US); Erika PAULSON, Berkeley, CA (US)

(73) Assignee: Conception Biosciences, Inc., Berkeley, CA (US)

(21) Appl. No.: 19/476,114

(22) PCT Filed: Apr. 19, 2024
(86) PCT No.: PCT/US2024/025567

§ 371 Date: Oct. 16, 2025

Related U.S. Application Data
(60) Provisional application No. 63/497,191, filed on Apr. 19, 2023.

(51) Int. Cl. C12N 5/071; C12N 5/075

(52) U.S. Cl. CPC C12N 5/0682; C12N 5/0609; C12N 2500/38; C12N 2506/45

(57) ABSTRACT

Provided in some aspects are methods for generating primordial follicles from human and other mammalian germ cells, with high efficiency, high cellular quality and high reproducibility. In some aspects, provided are methods and culture system for generating ovarian organoids, such as cell aggregates composed of both somatic and germ cells from primary and/or stem cell-derived sources, which are competent to progress primordial germ cells to oocytes that enter meiosis and are competent for follicle assembly. In some aspects, provided embodiments include a step to culture ovarian organoids in a fully defined serum-free media and in conditions with low defined serum protein components overall.

### CROSS-REFERENCE TO RELATED APPLICATIONS

This application claims priority to U.S. Provisional Application No. 63/497,191, filed Apr. 19, 2023, entitled “METHODS AND COMPOSITIONS FOR OVARIAN ORGANOID CULTURE,” the contents of which are incorporated by reference in their entirety.

### FIELD

The present disclosure relates generally to methods and compositions for generating ovarian organoids with defined cell types and morphological characteristics.

### BACKGROUND

In vitro derivation of gonadal cell populations and organoids, for example from stem cells, has the potential to provide powerful tools for studying biology and providing therapies and treatments for conditions associated with these reproductive tissues. However, the ability to derive and differentiate various gonadal cell types in culture remains limited. There is a need for new and improved methods to overcome these challenges. The present disclosure addresses these and other needs.

### SUMMARY

In some aspects, provided herein is a method of producing primordial follicles, the method comprising: a) providing a cell mixture of germ cells and somatic cells in culture, and b) culturing the cell mixture for a period of time in a defined medium to produce the primordial follicles, wherein the defined medium comprises protein or protein replacement, and the total protein or protein replacement concentration in the defined medium is less than 3.5 milligrams per milliliter (mg/mL). In some of any of the provided embodiments, each primordial follicle comprises (i) an oocyte, and (ii) a plurality of granulosa cells. In some aspects, provided herein is a method of producing oogonia and/or oocytes, the method comprising: a) providing a cell mixture of germ cells and somatic cells in culture, and b) culturing the cell mixture for a period of time in a defined medium to produce the oogonia and/or ooctyes, wherein the defined medium comprises protein or protein replacement, and the total protein or protein replacement concentration in the defined medium is less than 3.5 milligrams per milliliter (mg/mL).

In some of any of the provided embodiments, the method comprises further culturing the oogonia and/or oocytes to produce primordial follicles. In some of any of the provided embodiments, the further culturing is performed in a medium that is the same or different from the defined medium.

In some of any of the provided embodiments, the defined medium comprises protein or protein replacement wherein the total protein or protein replacement concentration in the defined medium is less than 3.5 mg/mL, less than 3.0 mg/mL, less than 2.5 mg/mL, less than 2.0 mg/mL, less than 1.5 mg/mL, less than 1.0 mg/mL, less than 0.5 mg/mL, or less than 0.1 mg/mL. In some of any of the provided embodiments, the total protein or protein replacement concentration in the defined medium is between about 0.1 mg/mL and 0.5 mg/mL, between about 0.5 mg/mL and 1.0 mg/mL, between about 1.0 mg/mL and 1.5 mg/mL, between about 1.5 mg/mL and 2.0 mg/mL, between about 2.0 mg/mL and 2.5 mg/mL, between about 2.5 mg/mL and 3.0 mg/mL, or between about 3.0 mg/mL and 3.5 mg/mL. In some of any of the provided embodiments, the total protein or protein replacement concentration in the defined medium is between about 0.1 mg/mL and 0.5 mg/mL. In some of any of the provided embodiments, the total protein or protein replacement concentration in the defined medium is between about 0.5 mg/mL and 1.0 mg/mL. In some of any of the provided embodiments, the total protein or protein replacement concentration in the defined medium is between about 1.0 mg/mL and 1.5 mg/mL. In some of any of the provided embodiments, the total protein or protein replacement concentration in the defined medium is between about 1.5 mg/mL and 2.0 mg/mL. In some of any of the provided embodiments, the total protein or protein replacement concentration in the defined medium is between about 2.0 mg/mL and 2.5 mg/mL. In some of any of the provided embodiments, the total protein or protein replacement concentration in the defined medium is between about 2.5 mg/mL and 3.0 mg/mL. In some of any of the provided embodiments, the total protein or protein replacement concentration in the defined medium is between about 3.0 mg/mL and 3.5 mg/mL. In some of any of the provided embodiments, the total protein or protein replacement concentration in the defined medium is at or about 0.1 mg/mL, 0.2 mg/mL, 0.3 mg/mL, 0.4 mg/mL, 0.5 mg/mL, 0.6 mg/mL, 0.7 mg/mL, 0.8 mg/mL, 0.9 mg/mL, 1.0 mg/mL, 1.5 mg/mL, 2.0 mg/mL, 2.5 mg/mL, 3.0 mg/mL, or 3.5 mg/mL, or a value between any of the foregoing.

In some of any of the provided embodiments, the total concentration of the protein or protein replacement in the defined medium is equal to or less than the concentration of protein in a medium comprising 7.5% (v/v) FBS. In some of any of the provided embodiments, the defined medium does not comprise serum. In some of any of the provided embodiments, the defined medium does not comprise FBS.

In some of any of the provided embodiments, the defined medium comprises one or more ingredients selected from: inorganic salts, sugars, amino acids, vitamins, organic acids, antioxidants, buffers; and the protein or protein replacement. In some of any of the provided embodiments, the defined medium comprises the protein or protein replacement in a base medium. In some of any of the provided embodiments, the base medium comprises Dulbecco's Modified Eagle's Medium (DMEM), Minimal Essential Medium (MEM), Basal Medium Eagle (BME), F-10, F-12, RPMI 1640, Glasgow's Minimal Essential Medium (GMEM), alpha Minimal Essential Medium (alpha MEM), advanced Minimal Essential Medium (advanced MEM), Iscove's Modified Dulbecco's Medium, or M199.

In some of any of the provided embodiments, the protein or protein replacement in the defined medium is a protein. In some of any of the provided embodiments, the protein or protein replacement in the defined medium is a protein replacement. In some of any of the provided embodiments, the protein comprises serum protein components. In some of any of the provided embodiments, the protein comprises mammalian serum protein components. In some of any of the provided embodiments, the serum protein components are defined. In some of any of the provided embodiments, the protein or protein replacement is or comprises albumin or an albumin substitute. In some embodiments, the protein is or comprises albumin. In some embodiments, the protein replacement is or comprises an albumin substitute. In a particular example, if the protein is albumin then a protein replacement is an albumin replacement. In some of any of the provided embodiments, the protein or protein replacement comprises one or more components selected from: bovine pituitary extract, plant hydrolysate (e.g., rice hydrolysate), an albumin, chick extract, bovine embryo extract, fetal calf albumin (fetuin), egg albumin, human serum albumin (HSA), other animal-derived albumins, or a bovine serum albumin. In some of any of the provided embodiments, the protein or protein replacement is or comprises a lipid enriched albumin. In some of any of the provided embodiments, the protein or protein replacement is or comprises a bovine serum albumin enriched with lipids. In some of any of the provided embodiments, the protein or protein replacement is or comprises AlbuMAX® lipid rich bovine serum albumin, optionally AlbuMAX® I lipid rich bovine serum albumin or AlbuMAX® II lipid rich bovine serum albumin. In some of any of the provided embodiments, the protein or protein replacement comprises a synthetic polymer. In some of any of the provided embodiments, the synthetic polymer is polyvinyl alcohol (PVA) and/or polyvinylpyrrolidone (PVP). In some embodiments, the protein replacement refers to any compound that can replace protein in the medium for use in the methods herein to give substantially similar results as protein. In some embodiments the protein or protein replacement in the defined media aid in cell attachment, growth, proliferation, and maintenance. In some of any of the provided embodiments, the defined medium is prepared by adding a protein or protein replacement to the base medium. In some of any of the provided embodiments, the protein or protein replacement is provided by a supplement medium that is added to the base medium. In some of any of the provided embodiments, the supplement medium is a defined serum replacement medium. In some of any of the provided embodiments, the supplement medium is added to the base medium to a final concentration of 7.5% (v/v) or less. In some of any of the provided embodiments, the supplement medium is added to the base medium to a final concentration of between about 1% and 7.5%, between about 1% and 5%, between about 1% and 3%, between about 3% and 5%, or between about 5% and 7.5%, all in v/v. In some of any of the provided embodiments, the supplement medium is added to the base medium to a final concentration of at or about 0.5%, at or about 1.0%, at or about 1.5%, at or about 2.0%, at or about 2.5%, at or about 3.0%, at or about 3.5%, at or about 4.0%, at or about 4.5%, at or about 5.0%, at or about 5.5%, at or about 6.0%, at or about 6.5%, at or about 7.0%, or at or about 7.5%, or a value between any of the foregoing, all percentages in v/v. In some of any of the provided embodiments, the supplement medium is added to the base medium to a final concentration of 2%. In some of any of the provided embodiments, the supplement medium comprises one or more ingredients selected from the group consisting of albumins or albumin substitutes, one or more amino acids, one or more vitamins, one or more transferrins or transferrin substitutes, one or more antioxidants, one or more insulins or insulin substitutes, one or more collagen precursors, and one or more trace elements. In some of any of the provided embodiments, the supplement medium comprises an albumin or albumin substitute. In some of any of the provided embodiments, the supplement medium comprises an antioxidant selected from the group consisting of reduced glutathione, ascorbic acid, and ascorbic acid-2-phosphate. In some of any of the provided embodiments, the supplement medium comprises a collagen precursor selected from the group consisting of L-proline and multimers or derivatives thereof, L-hydroxyproline multimers or derivatives thereof, and ascorbic acid or multimers thereof. In some of any of the provided embodiments, the supplement medium comprises a transferrin substitute that is an iron chelate, such as, for example, an iron chelate selected from the group consisting of a ferric citrate chelate and a ferrous sulfate chelate, optionally ferrous sulphate-7 water-EDTA. In some of any of the provided embodiments, the supplement medium comprises a insulin substitute selected from the group consisting of zinc chloride, zinc bromide, and zinc sulfate-7 water. In some of any of the provided embodiments, the supplement medium comprises an amino acid ingredient that comprises one or more amino acids selected from the group consisting of glycine, L-alanine, L-asparagine, L-cysteine, L-aspartic acid, L-glutamic acid, L-phenylalanine, L-histidine, L-isoleucine, L-lysine, L-leucine, L-glutamine, L-arginine, L-methionine, L-proline, L-hydroxyproline, L-serine, L-threonine, L-tryptophan, L-tyrosine, and L-valine, and derivatives thereof. In some of any of the provided embodiments, the the supplement medium comprises a trace element ingredient comprising one or more trace element moieties selected from the group consisting of Ag⁺, Al³⁺, Ba²⁺, Cd²⁺, Co²⁺, Cr³⁺, Ge⁴⁺, Se⁴⁺, Br⁻, I⁻, Mn²⁺, F⁻, Si⁴⁺, V⁵⁺, Mo⁶⁺, Ni²⁺, Rb⁺, Sn²⁺, and Zr⁴⁺. In some of any of the provided embodiments, the supplement medium comprises lipid-rich albumin (AlbuMAX), L-glycine, L-histidine, L-isoleucine, L-methionine, L-phenylalanine, L-proline, L-hydroxyproline, L-serine, L-threonine, L-tryptophan, L-tyrosine, L-valine, thiamine, reduced glutathione, L-ascorbic acid-2-phosphate, iron saturated transferrin, insulin, sodium selenite, Ag⁺, Al³⁺, Ba²⁺, Cd²⁺, Co²⁺, Cr³⁺, Ge⁴⁺, Se⁴⁺, Br⁻, I⁻, Mn²⁺, F⁻, Si⁴⁺, V⁵⁺, Mo⁶⁺, Ni²⁺, Rb⁺, Sn²⁺, and Zr⁴⁺.

In some of any of the provided embodiments, the supplement medium is knockout™ serum replacement (KSR). In some of any of the provided embodiments, the defined medium is a base medium supplemented with 1-5% defined knockout™ serum replacement (KSR). In some of any of the provided embodiments, the defined medium is a base medium supplemented with about 2% defined knockout™ serum replacement (KSR).

In some of any of the provided embodiments, the cell mixture is or forms an ovarian organoid. In some of any of the provided embodiments, the ovarian organoid is cultured in a fully immersed suspension culture. In some of any of the provided embodiments, the ovarian organoid is cultured at an air-liquid interface using permeable culture membranes. In some of any of the provided embodiments, the method further comprises removing the defined medium from the cell mixture after oogonia and/or oocytes are produced. In some of any of the provided embodiments, the ovarian organoid is composed of 500-300,000 cells. In some of any of the provided embodiments, the percentage of germ cells in the ovarian organoid is between 1% and 20%, or between 1% and 50%, of the total number of cells in the organoid.

In some of any of the provided embodiments, the cell mixture is cultured for between 1 day and 300 days in vitro.

In some of any of the provided embodiments, the method further comprises activating the primordial follicles to produce primary follicles. In some of any of the provided embodiments, the germ cells are primordial germ cells (PGCs). In some of any of the provided embodiments, the germ cells are primordial germ cell-like cells PGCLCs. In some of any of the provided embodiments, the germ cells express one or more genes selected from: TFAP2C, PRDM1, and POU5F1. In some of any of the provided embodiments, the germ cells are human. In some of any of the provided embodiments, the germ cells are derived from in vivo tissue. In some of any of the provided embodiments, the germ cells are derived from pluripotent stem cells (PSCs). In some embodiments, the PSCs are induced pluripotent stem cells (iPSCs). In some of any of the provided embodiments, the germ cells differentiate into the oogonia and/or oocytes.

In some of any of the provided embodiments, the oogonia express one or more genes selected from: DDX4, DAZL, STRA8, SYCP3, and SYCP1. In some of any of the provided embodiments, the oocytes express one or more genes selected from FIGLA and ZP3.

In some of any of the provided embodiments, the somatic cells are mammalian ovarian somatic cells. In some of any of the provided embodiments, the somatic cells are collected in vivo. In some of any of the provided embodiments, the somatic cells are collected from in vivo fetal mammalian ovaries. In some of any of the provided embodiments, the somatic cells are from human, non-human primate, pig, rabbit, cow, mouse, rat, donkey, and/or rabbit. In some of any of the provided embodiments, the somatic cells are differentiated in vitro from another cell type. In some of any of the provided embodiments, the somatic cells are derived from pluripotent stem cells (PSCs). In some embodiments, the PSCs are induced pluripotent stem cells (iPSCs). In some of any of the provided embodiments, the somatic cells comprise one or more ovarian somatic cell types. In some of any of the provided embodiments, one or more of the somatic cell types expresses one or more genes characteristic of intermediate mesoderm, including WT1. In some of any of the provided embodiments, one or more of the somatic cell types expresses one or more genes characteristic of coelomic epithelium, including WT1 and GATA4. In some of any of the provided embodiments, one or more of the somatic cell types expresses one or more genes characteristic of granulosa cells, including WT1, GATA4, LHX9, NR5A1 and/or FOXL2. In some of any of the provided embodiments, one or more of the somatic cell types expresses one or more genes characteristic of bipotential gonad, including WT1, GATA4, LHX9 and/or NR5A1 .

In some aspects, provided herein is a method of generating an ovarian organoid comprising one or more primordial follicles, wherein: each primordial follicle comprises (i) an oocyte, and (ii) a plurality of granulosa cells, wherein the oocyte is derived from a human or non-human primate precursor. In some of any of the provided embodiments, the oocyte is derived from a human germ cell. In some embodiments, the human germ cell is derived from a stem cell. In some of any of the provided embodiments, the ovarian organoid comprises at least 5 primordial follicles, at least 10 primordial follicles, at least 20 primordial follicles, at least 50 primordial follicles, at least 100 primordial follicles, at least 500 primordial follicles, or more that 500 primordial follicles. In some of any of the provided embodiments, the ovarian organoid is derived from a cell mixture of ovarian germ cells and somatic cells in culture, and the ovarian organoid comprises at least 5, at least 10, at least 50, at least 100, or at least 500 oocytes and/or oogonia cells derived from the ovarian germ cells, wherein the ovarian germ cells are from human or non-human primate. In some of any of the provided embodiments, the ovarian germ cells are human germ cells. In some of any of the provided embodiments, the ovarian germ cells are primary germ cells (PGCs) or primary germ cell-like cells (PGCLCs). In some of any of the provided embodiments, the percentage of oocytes, oogonia, and/or other germ cells in the ovarian organoid is about or more than 1%, about or more than 5%, about or more than 10%, about or more than 20%, about or more than 30%, about or more than 40%, or about or more than 50% of the total number of cells in the ovarian organoid.

### BRIEF DESCRIPTION OF THE DRAWINGS

FIG. 1 shows exemplary results from culturing ovarian organoids in fully defined medium with 2% (v/v) defined serum replacement (DSR) (e.g., 2% KSR) showing significantly improved PGC survival after 21 days in organoid culture compared to culture in 10% DSR, and also in comparison to standard published methods, i.e., culturing in long-term media containing 2% or 10% (v/v) FBS. Left panels: histological sections of ovarian organoids cultured in media containing 2% (v/v) DSR, 2% (v/v) FBS, 10% (v/v) DSR, or 10% (v/v) FBS. White arrows mark surviving PGCs, which are positively co-stained for a hPSC-derived reporter marker and the PGC marker AP2g. Structures denoted by an asterisk are hPSC-derived cells that have lost their PGC identity. Right panel: quantification of the average number of surviving of PGCs in 3 separate histological sections at day 21 for each group.

FIG. 2 shows exemplary images demonstrating robust survival of PSC-derived germ cells in ovarian organoids cultured in a defined medium (long-term media comprising a defined serum replacement (e.g., KSR)). Ovarian organoids were generated by mixing human iPSC-derived female germ cells derived from a fluorescently tagged reporter cell line with ovarian somatic cells. Images show brightfield (imaging all cells; top left of each panel) and fluorescence to visualize fluorescently-tagged PSC-derived germ cells (top right of each panel) separately and superimposed (large image for each panel) over time in culture with either defined medium (2% (v/v) DSR) or with 10% (v/v) FBS conditions.

FIG. 3A-FIG. 3C shows exemplary images demonstrating generation of PSC-derived female germ cells that are appropriately advanced for competency of follicle assembly. Prior to follicle assembly in the developing mammalian gonad, female germ cells undergo development through defined stages of germ cell progression. After meiotic initiation and subsequent meiotic arrest, female germ cells begin expressing markers including LMOD3 and ZP3, signaling their competency for follicle assembly. FIG. 3A shows a histological section of a 17 gestational week human fetal ovary, with germ cells expressing LMOD3 in the cytoplasm and ZP3 along the periphery of the cell. FIG. 3B shows a histological section of an ovarian organoid, cultured long term in defined serum conditions. Many cells within this section express LMOD3 in the cytoplasm and ZP3 along the periphery of the cells. A nuclear reporter shows that these cells are PSC-derived. FIG. 3C shows ovarian organoids cultured for long term with 10% (v/v) FBS serum conditions. Few to no cells in this group express LMOD3 or ZP3. Far fewer cells overall are positive for the PSC-derived reporter.

FIG. 4A and FIG. 4B shows exemplary results demonstrating that defined serum conditions lead to robust survival, progression and follicle assembly of PSC-derived germ cells in ovarian organoids compared to 10% (v/v) FBS conditions. The left and right panels of FIG. 4A show histological sections of an ovarian organoid generated with human PSC-derived germ cells and ovarian somatic cells cultured long term in defined serum conditions (left) or 10% (v/v) FBS conditions (right), stained for oogonia marker DDX4, granulosa somatic marker FOXL2, and a cytoplasmic reporter for PSC-derived cells. The organoid cultured in defined serum conditions contains a robust number of oogonia and granulosa cells that are beginning to organize into primordial follicles. The organoid cultured in 10% (v/v) FBS conditions contains large areas that are completely acellular, with very low survival of PSC-derived germ cells, and few FOXL2+granulosa cells. FIG. 4B shows the same histological sections with the fluorescent markers separated into individual channels.

FIG. 5 shows exemplary results demonstrating efficient derivation of primordial follicles from PSC-derived germ cells in an in vitro ovarian organoid in defined serum conditions. Top left panel shows a histological section of a human fetal ovary at 19 weeks gestation, stained with oogonia/oocyte marker DDX4 in the cytoplasm of germ cells, while nuclear FOXL2 staining marks the surrounding somatic granulosa cells. The two panels on the right show a histological section of an ovarian organoid cultured long term in defined serum conditions, with DDX4 staining oogonia/oocytes in the cytoplasm and FOXL2 staining the nuclei of surrounding somatic granulosa cells. On the far right, a cytoplasmic reporter defines cells that are PSC-derived. All PSC-derived cells within the organoid are DDX4+ and surrounded by organizing FOXL2+ granulosa cells. The bottom three rows of panels are selected zoomed in images of examples of follicle structures seen within larger sections of ovarian organoids cultured long term in defined serum conditions: the bottom panels are the cytoplasmic reporter that defines cells that are PSC-derived; the middle panels are the nuclear FOXL2 staining; the top panels are an overlay of DDX4 and FOXL2 staining from the panels in the two rows below. Cells denoted by an asterisk are DDX4+ germ cells.

FIG. 6A-FIG. 6C show uniform manifold approximation and projection (UMAP) graphs of single-cell RNA-sequencing data comparing in vivo control human fetal germ cells from 6-22 post conception weeks (pwc), in vitro human pluripotent stem cell-derived germ cells from primordial germ cell like cell (PGCLC) starting cultures in the cell mixture, and/or the in vitro germ cells after culture of the ovarian organoid in the defined medium containing defined serum replacement (e.g., KSR). FIG. 6A shows exemplary results demonstrating that the in vivo control human fetal germ cells span stage 1 to stage 4 of development. FIG. 6B shows exemplary results of single-cell RNA-sequencing data of the in vivo control human fetal germ cells merged with human pluripotent stem cell-derived germ cells from PGCLC starting cultures in the cell mixture. FIG. 6C shows exemplary results of single-cell RNA-sequencing data of the in vivo control human fetal germ cells merged with germ cells obtained from an ovarian organoid. After culture of the ovarian organoids in the defined medium, a subset of pluripotent stem cell-derived in vitro cultured human germ cells in the ovarian organoid overlaped directly with human fetal germ cells spanning from stage 1 to stage 4, indicating in vitro cultured germ cells progress in this culture system. Single cell RNA-seq was performed using the 10x Genomics platform.

FIG. 7 shows a control human fetal ovary control (top panels) and a cultured ovarian organoid (bottom panels) stained for a germ cell marker (DDX4+) which stains the cell periphery (subset of staining indicated by arrows in the overlay images) and with nuclear SYCP3+ staining (subset of staining indicated by arrows in the overlay images), which is an indicator of meiotic entry.

### DETAILED DESCRIPTION

Provided herein are in vitro methods of producing follicles, such as primary or primordial follicles, and oogonia and/or oocytes. In some of the provided methods, the follicles (e.g., primordial follicles) are generated by culturing somatic cells and germ cells in a cell mixture. For example, the cells are cultured in a defined medium, particularly a defined medium with a low total protein concentration, wherein the germ cells and the somatic cells in the cell mixture aggregate to form an ovarian organoid, and also embodiments where the germ cells in the ovarian organoid further differentiate into oogonia and/or oocytes. In other embodiments, culture of the formed ovarian organoid in the defined medium further produce follicles, such as primary or primordial follicles, including follicles comprising an oocyte surrounded and/or contacted by a plurality of granulosa cells, e.g., somatic cells. Thus, the present methods provide conditions, including media preparations, for supporting follicle development and oogonia and oocyte maturation in vitro.

For example, it is shown herein that culture of a mixture of germ cells and somatic cells in a defined medium lacking serum and comprising a low concentration of protein or protein replacement produces primordial follicles, oogonia, and oocytes. In provided embodiments, primordial follicles, oogonia, and oocytes can be produced in culture conditions where the concentration of the total protein or protein replacement in the defined culture medium is less than 3.5 mg/mL.

The low percentage or concentration of protein or protein replacement in the defined medium provided herein can provide benefits for the culture of the desired cell types compared to a higher percentage of protein or compared to serum. For example, the benefits of a low percentage of protein or protein replacement in the defined medium can include improved survival of the germ cells, reduction of off target growth from undefined medium, and improved germ cell progression for the production of primordial follicle development. As shown herein, a defined medium with a low concentration or percentage of protein or protein replacement can support improved generation of follicles, such as primordial follicles, compared to culture conditions with media used in published protocols for ovarian organoid culture, such as media comprising FBS (e.g. 10% (v/v) FBS). Follicle generation in a defined medium comprising low protein or protein replacement, such as at or about 3.5 mg/mL total protein or protein replacement, can produce increased follicle generation compared to culture in the same medium but containing a higher amount of protein or protein replacement or serum.

During mammal fetal development, germ cells are developed in the gonads. The gonads are the organs that produce gametes (sperm and eggs) in males and females. In the early stages of fetal development, the gonads are undifferentiated and have the potential to develop into either testes or ovaries.

The development of germ cells in the fetal gonad begins with the migration of primordial germ cells (PGCs) from the yolk sac to the developing gonad. PGCs are the precursor cells of eggs and sperm, and they arise early in fetal development. PGCs are first specified in the epiblast, which is the outer layer of the blastocyst. They then migrate to the developing gonads, guided by signals from surrounding tissues.

Once PGCs reach the gonads, they begin to interact with the surrounding somatic cells, which are the non-germ cells of the gonads. The somatic cells provide signals that instruct the germ cells to differentiate and begin meiosis, which is the process of cell division that produces haploid gametes.

In females, the somatic cells of the developing ovaries promote the differentiation of the germ cells into oogonia, a cell committed to become an egg. During division, oogonia form a structure called nests, which are surrounded by granulosa cells. As development progresses, oogonia begin to differentiate into oocytes, which are arrested in the first meiotic division. At this point, a process called nest breakdown starts, wherein granulosa cells migrate between the oocytes and reorganize creating structures called primordial follicles. These follicles stay dormant until puberty, when some of them start activating again. Once active, the follicles, and the oocyte inside, start growing in size. Once reaching a certain size, oocytes are ready to mature into eggs that can then be fertilized to produce embryos.

Some portions of this process can be replicated in vitro, whereby mammalian primordial germ cells can be derived from pluripotent stem cells using various techniques. Multiple protocols exist, which, although differing slightly between species, primarily utilize BMP signaling to differentiate mammalian pluripotent stem cells (PSCs) into PGCs (Irie et al., 2015; Sasaki et al., 2015; Kobayashi et al., 2021; Sakai et al., 2020; Seika et al., 2020). Some of these methods may include an intermediate stage, while others involve the overexpression of specific transcription factors or genes from the GATA family (Hamazaki et al., 2020; Kojima et al., 2021; Kramme et al., 2022).

In mice, PGCs, either in vitro or in vivo-derived, can be further developed into oogonia and oocytes, and ultimately form follicles with the help of fetal ovarian somatic cells from in vivo or in vitro sources (Hikabe et al., 2016; Yoshino et al., 2021). A similar model has been used to progress human and non-human primate-derived PGCs into oogonia, and even to a meiotic stage, by co-culturing with mouse fetal ovarian somatic cells in a high-serum content culture method. However, this differentiation process remains highly inefficient, with the majority of germ cells dying and only a few able to progress (Yamashiro et al., 2018; Murase et al., 2020; Gyobu-Motani et al., 2023). To date, no previous system has been able to robustly progress high numbers of human cells to the oogonia and meiotic stage, and no previous system has been able at all to progress in vitro derived human germ cells into the oocyte or follicle stage, a critical step towards generating eggs from somatic cells in vitro.

The methods provided herein overcome previous limitations and can be used for preparing ovarian organoids in vitro. In some aspects, the provided compositions and methods address the lack of survival of human germ cells within ovarian organoids in previous culture methods. In some aspects, the provided methods, which include culture in a lower amount of protein or protein replacement than conventional methods, and compositions induce robust germ cell progression into oocytes that have the ability to form primordial follicles. In some aspects, the provided methods and compositions induce robust generation of ovarian primordial follicles that closely resemble in vitro follicles of late first trimester and adult ovarian primordial follicles. In some aspects, the provided methods and compositions include using a defined medium for long-term culture of ovarian organoids. In some aspects, the defined medium is a fully defined medium that contains an overall low percentage or concentration of protein and/or protein replacement components.

As described herein, the low percentage or concentration of protein or protein replacement in the defined medium provided herein can improve the survival of the germ cells, reduce off target growth from undefined medium, and allow germ cell progression to allow for the rapid and robust production of primordial follicle development. Culturing germ cells in medium that does not contain serum and that comprises this low level of protein, which is decreased compared to conventional methods, results in increased cell survival after a prolonged time in culture (e.g., 21 days). For example, as shown herein, lowering the amount of protein in the cell culture medium can increase survival of PGCLCs and germ cells. The defined medium as used herein does not contain serum and contains less protein (e.g., at or about 3.5 mg/mL) or a protein replacement compared to conventional methods that are cultured in the presence of serum, which contains a mixture of proteins. In some aspects, culturing ovarian organoids in the defined medium vastly improves germ cell survival, proliferation, differentiation, and formation of follicles. In some aspects, the improvement is compared to current standard culture conditions that rely on culture in media containing fetal bovine serum.

Also provided are ovarian organoids, such as ovarian organoids produced using any of the methods or compositions provided herein. In some embodiments, the ovarian organoids comprise germ cells, such as oogonia and/or oocytes derived from precursors, such as primordial germ cells. In some embodiments, the germ cells (e.g. the primordial germ cells) are in vitro-derived, such as from a pluripotent stem cell(s) (PSC), such as induced pluripotent stem cells (iPSCs) or embryonic stem cells (ESCs). In some embodiments, the ovarian organoids comprise ovarian somatic cells, which can support germ cell survival, proliferation, and/or differentiation. In some embodiments, the ovarian organoids comprise follicles, such as primordial follicles. In some embodiments, the follicles, such as primordial follicles, can be further progressed (e.g. activated).

In some aspects, provided herein is a method of producing primordial follicles. In some embodiments, the method comprises: a) providing a cell mixture of germ cells and somatic cells in culture, and b) culturing the cell mixture for a period of time in a defined medium to produce the primordial follicles. In some aspects, provided herein is a method of producing oogonia and/or oocytes. In some embodiments, the method comprises: a) providing a cell mixture of germ cells and somatic cells in culture, and b) culturing the cell mixture for a period of time in a defined medium to produce the oogonia and/or ooctyes. In some embodiments, the defined medium comprises protein or protein replacement, and the total protein or protein replacement concentration in the defined medium is less than 3.5 milligrams per milliliter (mg/mL).

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Ovarian Organoid Culture

In some embodiments, provided are methods for culturing gonadal cells, such as ovarian cells, in vitro (e.g. in culture). In some embodiments, the methods comprise culturing germ cells and somatic cells together to form a cell mixture. In some embodiments, the cell mixture is cultured in a defined medium, such as any defined medium provided herein and/or described in Section II and in the Examples. In some embodiments, the cell mixture is cultured in a defined medium that does not comprise any serum and that comprises a low concentration or percentage of protein or protein replacement.

In some embodiments, the methods comprise culturing germ cells and somatic cells together to form a cell mixture in a defined medium comprising a protein or protein replacement, and the total concentration of the protein or protein replacement in the defined medium is less than 3.5 mg/mL in the absence of serum. In some examples, the cell mixture of germ cells and somatic cells can be further cultured in defined medium containing a protein or protein replacement at less than 3.5 mg/mL total to form ovarian organoids. In some embodiments, the ovarian organoids are cultured in the defined medium comprising the low concentration (e.g., less than about 3.5 mg/mL) of total protein or protein replacement until oocytes and/or oogonia are derived. In some embodiments, following the ovarian organoids prepared from culturing the germ cells and somatic cells in the culture medium containing low amounts of protein or protein replacement are further cultured until follicles are formed. In some embodiments, after ovarian organoids are formed in the low protein or protein replacement culture, the ovarian organoids are further cultured in the defined media or can be cultured in a different medium. In embodiments herein, culture of germ cells, somatic cells, and/or ovarian organoids in the defined medium as described herein, that contains less protein than standard culture media preparations, increases survival, proliferation, and differentiation of germ cells, to support generation of oocytes, oogonia, and/or follicles (e.g. primordial follicles) in the ovarian organoid culture.

In some aspects, provided herein are methods of producing primordial follicles, such as from culturing ovarian cells in vitro. In some embodiments, the ovarian cells are cultured as ovarian organoids. In some embodiments, an ovarian organoid is a mixture of aggregated cell types comprising one or more ovarian cell types, such as any of the ovarian cell types described herein, including ovarian germ cells or somatic cells. In some embodiments, the ovarian organoids are cultured in long-term culture, for example for 1-300 days in vitro.

In some embodiments, the methods described herein provide for efficient production of ovarian germ cells, such as oocytes and/or oogonia. In some embodiments, the methods allow for the production of follicles, such as primordial follicles. In some embodiments, primordial follicles comprise an oocyte surrounded and/or contacted by a plurality of granulosa cells. In some embodiments, primordial follicles can be further activated, for example to produce primary follicles.

In some embodiments, an ovarian organoid is a mixture of aggregated cell types comprising one or more ovarian cell types, such as any of the ovarian cell types described herein, such as germ cells, as described herein and/or somatic cells, as described in Section I.B. In some embodiments, the ovarian organoid culture or the ovarian organoids comprise germ cells.

In some embodiments, ovarian organoids comprise germ cells and/or somatic cells.

In some embodiments, the germ cells and/or somatic cells are mammalian cells. The germ cells and/or somatic cells in embodiments herein can be from various sources, including but not limited to human, pig, rabbit, cow, mouse, rat, donkey, rabbit, non-human primate, or other mammalian species. The germ cells and somatic cells need not be the same species. In some embodiments, the germ cells are human. In some embodiments, the somatic cells are human. In some embodiments the germ cells and somatic cells are human. In some embodiments, the somatic cells are not human.

In some embodiments, the germ cells and/or somatic cells are derived from stem cells. In some embodiments, the germ cells and/or somatic cells are derived from stem cells such as pluripotent stem cells (PSCs), embryonic stem cells, or induced pluripotent stem cells (iPSCs). In some embodiments, the germ cells and/or somatic cells are derived from stem cells that are pluripotent stem cells. Various sources of pluripotent stem cells can be used in the methods herein, including embryonic stem(ES) cells and induced pluripotent stem cells (iPSCs). In some embodiments, pluripotent stem cells are induced pluripotent stem cells (iPSCs), artificially derived from a non-pluripotent cell. In some embodiments, a non-pluripotent cell is a cell of lesser potency to self-renew and differentiate than a pluripotent stem cell. iPSCs can be generated by a process known as reprogramming, wherein non-pluripotent cells are effectively “dedifferentiated” to an embryonic stem cell-like state by engineering them to express genes such as, for example, OCT4, SOX2, and KLF4 (Takahashi and Yamanaka Cell (2006) 126:663-76).

In some embodiments, the germ cells and/or somatic cells are derived from embryonic stem cells. In some embodiments, the germ cells and/or somatic cells are derived from iPSCs.

In some embodiments, the germ cells and/or somatic cells are isolated from in vivo tissue. In some embodiments, the germ cells and/or somatic cells are isolated from primary tissue. In some embodiments, the germ cells and/or somatic cells are isolated from primary ovarian tissue. In some embodiments, the germ cells (e.g., PGCs or PGCLCs), and/or somatic cells are isolated from primary ovarian tissue. In some embodiments, primary ovarian tissue is dissected and kept in culture.

### A. Germ Cells

In some embodiments, germ cells can be any cells that can differentiate into a reproductive cell, including but not limited to, a primordial germ cell, a primordial germ cell-like cell, an oogonia, and/or an oocyte, or a cell at any developmental stage in between any of the foregoing. In some embodiments, the germ cells and any somatic cells described herein, such as those described in Section I.B. are cultured together to form a cell mixture, such as any described in Section I.C. In some embodiments, the cell mixture aggregates to form an ovarian organoid and in some aspects primary follicles.

In some embodiments, the germ cells are human germ cells. In some embodiments, the germ cells comprise primordial germ cells (PGCs) or primordial germ cell-like cells (PGCLCs).

In some embodiments, the germ cells (e.g., PGCs or PGCLCs) are derived from stem cells, such as pluripotent stem cells (PSCs), embryonic stem cells, or induced pluripotent stem cells (iPSCs), as described in Section I. In some embodiments, the germ cells (e.g., PGCs or PGCLCs) are derived from embryonic stem cells. In some embodiments, the germ cells (e.g., PGCs or PGCLCs) are derived from iPSCs. In some embodiments, the germ cells, such as PGCs or PGCLCs, are in vitro derived. Exemplary in vitro methods for generating PGCLCs or PGCs from iPSCs or ESCs are provided in e.g., Vijayakumar et al., Nat Commun (2023) 14, 5690,; Irie et al., Cell (2015), 160(1-2):253-68; Irie et al., Methods Mol Biol (2017) 1463:217-226; Jo et al., eLife (2022) 11:e72811; Overeem et al., Cell Rep Methods (2023) 3(6):100488; Sasaki et al., Cell Stem Cell, 17(2):178-94; Esfahani et al., Nat Comm. (2024) 2;15(1):167; Chen et al., Nat Methods (2011) 8, 424-429, the disclosure of each of which is incorporated by reference herein in its entirety. In some embodiments, pluripotent stem cells are cultured in media containing a variety of supplements (e.g., FGF, BMP2, BMP4 or a WNT inhibitor) that drive differentiation of the stem cell(s) to germ cells (e.g., PGCs or PGCLCs). Following differentiation in culture, in vitro derived PGCs or PGCLCs can be isolated by mechanical dissection, dissociation, and/or isolation. In some embodiments, the in vitro derived PGCs or PGCLCs can be isolated using methods such as fluorescence activated cell sorting (FACS) or magnetic activated cell sorting (MACS). In some embodiments, in vitro derived germ cells (e.g., PGCs or PGCLCs) are isolated by FACS using CD38 as a cellular marker.

In some embodiments, the germ cells (e.g., PGCs or PGCLCs) are isolated from in vivo tissue, e.g., from primary tissue. In some embodiments, the germ cells (e.g., PGCs or PGCLCs) are isolated from primary ovarian tissue. Exemplary methods for obtaining PGCLCs or PGCs from ovarian tissue are provided in e.g., Hayashi et al., Nat Protoc (2013) 8(8):1513-24, the disclosure of which is incorporated by reference herein in its entirety.

In some embodiments, germ cells can be collected from in vivo tissue. In some embodiments, germ cells can be collected from fetal ovary tissue. In some examples the germ cells are collected from fetal ovary tissue from a fetus between 6 and 22 weeks of gestational age. For example, bovine fetal ovary tissue can be collected between 6 and 22 weeks of gestational age, pig fetal ovary tissue can be collected between 6 and 22 weeks of gestational age, non-human primate fetal ovary tissue can be collected between 6 and 22 weeks of gestational age, and mouse fetal ovary tissue can be collected between 10 and 21 days of gestational age. Fetal tissue can be isolated and prepared by methods known in the art. For example, in general, fresh tissue is divided by mincing, teasing, comminution, and/or collagenase digestion. The desired cells can be isolated from contaminating cells and materials by washing, filtering, centrifuging and/or picking procedures, and optionally cultured and/or cryopreserved as desired prior to encapsulation. In some embodiments, primary ovarian tissue is dissected and kept in culture. In some examples, after tissue dissection, the ovaries are chemically and mechanically dissociated down to a single cell suspension. Following generation of the single-cell suspension, PGCs or PGCLCs can be isolated by mechanical dissection, dissociation, and isolation, e.g. using protocols such as fluorescence activated cell sorting (FACS) or magnetic activated cell sorting (MACS). In some embodiments, native germ cells (e.g., PGCs or PGCLCs) are isolated by FACS.

In some embodiments, the germ cells are isolated from in vivo tissue. In some embodiments, the germ cells are stem cell derived. In some embodiments, the germ cells are a mix of cells isolated from in vivo tissue and germ cells derived from stem cells. In some embodiments, the germ cells are not a mix of cells isolated from in vivo tissue and germ cells derived from stem cells.

In some embodiments, various germ cell populations can be defined and/or identified by any suitable marker (e.g. an expressed gene or reporter). In some embodiments, germ cells can be defined and/or identified by primordial germ cell marker genes. In some embodiments, the germ cells express one or more primordial germ cell marker genes. Primordial germ cell marker genes include, but are not limited to, NANOS3, TFAP2C, SOX17, PRDM1, POU5F1, OCT4, NANOG, TFCP2L1, and TBXT. In some embodiments, the germ cells express one or more of NANOS3, TFAP2C, SOX17, PRDM1, POU5F1, NANOG, and TBXT primordial germ cell marker genes. In some embodiments, the germ cells express one or more of TFAP2C, PRDM1, and POU5F1 primordial germ cell marker genes. In some embodiments, the germ cells express NANOS3, TFAP2C, SOX17, PRDM1, POU5F1, NANOG, and TBXT primordial germ cell marker genes. In some embodiments, the germ cells express one or more of NANOS3, TFAP2C, SOX17, PRDM1, POU5F1, NANOG, and TBXT where at least or at least about 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97%, or 98% of the germ cells express one or more of NANOS3, TFAP2C, SOX17, PRDM1, POU5F1, NANOG, and TBXT. In some embodiments, the germ cells express TFAP2C, PRDM1, and POU5F1 primordial germ cell marker genes. In some embodiments, at least about any one of: 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97%, or 98% of the germ cells express TFAP2C, PRDM1, and POU5F1. In some embodiments, at least about 80% of the germ cells express TFAP2C, PRDM1, and POU5F1. In some embodiments, at least about 90% of the germ cells express TFAP2C, PRDM1, and POU5F1. In some embodiments, at least about 95% of the germ cells express TFAP2C, PRDM1, and POU5F1.

In some embodiments, the germ cells do not express alternative lineage markers. Exemplary alternative lineage markers include, but are not limited to, FOXA2, HHEX, CDX2, and SOX2. In some embodiments, the germ cells do not express one or more of the alternative lineage markers FOXA2, HHEX, CDX2, and SOX2. In some embodiments, the germ cells do not express FOXA2, HHEX, CDX2, and SOX2. In some embodiments, the germ cells do not express one or more of FOXA2, HHEX, CDX2, and SOX2 where at least or at least about 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97%, or 98% of the germ cells do not express one or more of FOXA2, HHEX, CDX2, and SOX2. In some embodiments, the germ cells do not express FOXA 2 and SOX 2. In some embodiments, at least about 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97%, or 98% of the germ cells do not express FOXA2 and SOX2. In some embodiments, at least about 50% of the germ cells do not express FOXA2 and SOX2. In some embodiments, at least about 60% of the germ cells do not express FOXA2 and SOX2. In some embodiments, at least about 70% of the germ cells do not express FOXA2 and SOX2. In some embodiments, at least about 80% of the germ cells do not express FOXA2 and SOX2. In some embodiments, at least about 90% of the germ cells do not express FOXA2 and SOX2. In some embodiments, at least about 95% of the germ cells do not express FOXA2 and SOX2.

In some embodiments, the germ cells express one or more of NANOS3, TFAP2C, SOX17, PRDM1, POU5F1, NANOG, and TEXT and/or do not express one or more of FOXA2 and SOX2. In some embodiments, the germ cells express NANOS3, TFAP2C, SOX17, PRDM1, POU5F1, NANOG, and TEXT and do not express FOXA2 and SOX2. In some embodiments, at least or at least about 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97%, or 98% of the germ cells express NANOS3, TFAP2C, SOX17, PRDM1, POU5F1, NANOG, and TEXT and/or do not express FOXA2 and SOX 2. In some embodiments, at least about 80% of the germ cells express NANOS3, TFAP2C, SOX17, PRDM1, POU5F1, NANOG, and TEXT and/or do not express FOXA 2 and SOX2. In some embodiments, at least about 90% of the germ cells express NANOS3, TFAP2C, SOX17, PRDM1, POU5F1, NANOG, and TEXT and/or do not express FOXA 2 and SOX2. In some embodiments, at least about 95% of the germ cells express NANOS3, TFAP2C, SOX17, PRDM1, POU5F1, NANOG, and TEXT and/or do not express FOXA2 and SOX2.

### B. Somatic Cells

In some embodiments, somatic cells can be any cell that forms the body of a multicellular organism other than a gamete and/or germ cell, such as ovarian somatic cells. In some embodiments, the ovarian somatic cells are mammalian. In some embodiments, the ovarian somatic cells are fetal ovarian somatic cells. In some embodiments, the fetal ovarian somatic cells can be from any suitable species that supports the development of the germ cells in the organoid. In some embodiments, the somatic cells and any germ cells provided herein, such as those described in Section I.A. are cultured together to form a cell mixture, such as any described in Section I.C. In some embodiments, the cell mixture aggregates to form an ovarian organoid and in some aspects primary follicles.

In some embodiments, the species of the somatic cell is the same as the germ cell (e.g. human germ cell and human somatic cell). The species of the somatic cell does not need to be the same as the germ cell (e.g. human germ cell and non-human somatic cell). For example, the species can be any suitable species, including human, pig, rabbit, cow, mouse, rat, donkey, rabbit, non-human primate, or other species. Somatic cells can be derived from a precursor cell type (e.g. derived in vitro from stem cells or other precursors). In other embodiments, somatic cells can be collected from in vivo tissue, such as fetal ovary tissue. For example, bovine fetal ovary tissue can be collected between 6 and 22 weeks of gestational age, pig fetal ovary tissue can be collected between 6 and 22 weeks of gestational age, non-human primate fetal ovary tissue can be collected between 6 and 22 weeks of gestational age, or mouse fetal ovary tissue can be collected between 10 and 21 days of gestational age.

In some embodiments, somatic cells are derived in vitro from stem cells. Exemplary in vitro methods for generating somatic cells from iPSCs or ESCs are provided in e.g., WO 2022/094628, WO 2022/2211054, and Yoshino et al., Science (2021) 373(6552) eabe0237, the disclosure of which is incorporated by reference herein in its entirety. In some embodiments, pluripotent stem cells are cultured in media containing one or more supplements that drive differentiation of the stem cell to somatic cells. In some embodiments, pluripotent stem cells are cultured in at least one or more media conditions containing one or more supplements, including but not limited to ActivinA, CHIR, BMP4, follistatin, EGF FGF, retinoic acid(RA), and/or FGF In some embodiments, the culture is a step-wise differentiation that involves culture that differentiates stem cells into various intermediates such as nascent mesoderm, intermediate mesoderm and then ovarian somatic cells (e.g., gonadal somatic cells). In some embodiments, the culture is a multi-step culture that goes through mesoderm and intermediate mesoderm progenitors. In some embodiments, the culture can be a 2D-culture. In some embodiments, the culture is a 3D-culture.

In some embodiments, the pluripotent stem cells are cultured in a first media, a second media, and/or a third media each containing one or more supplements, including but not limited to one or more of ActivinA, CHIR, BMP4, follistatin, EGF FGF, RA, and/or FGF for the generation of somatic cells. In some embodiments, the culture into mesoderm uses CHIR. In some embodiments, the culture into intermediate mesoderm uses RA and one or more other molecules. In some embodiments, pluripotent stem cells, such as embryonic stem cells (ESCs) are cultured for a suitable amount of time to produce somatic cells. Following differentiation in culture, in vitro derived somatic cells can be isolated by mechanical dissection, dissociation, and isolation, e.g. using protocols such as fluorescence activated cell sorting (FACS) or magnetic activated cell sorting (MACS).

Any of the cells in the ovarian organoid, such as ovarian germ cells or ovarian somatic cells, can be obtained or derived by any suitable means. For example, the cells can be obtained by mechanical dissection, dissociation, and/or isolation, e.g. using protocols such as fluorescence activated cell sorting (FACS) or magnetic activated cell sorting (MACS). In some embodiments, cells can be derived from another cell type, e.g., by differentiation from a precursor cell, or by any suitable means. For example, cells can be generated in vitro from any other cell, e.g., by activating cell signaling pathways, transcription factor overexpression, or other suitable methods.

The somatic ovarian cell types can comprise one or more somatic ovarian cell types, such as intermediate mesoderm, coelomic epithelium, granulosa cells, and bipotential gonad. In some embodiments, the ovarian somatic cells can comprise or comprise characteristics of intermediate mesoderm, such as expression of the gene WT1. In some embodiments, the ovarian somatic cells can comprise or comprise characteristics of coelomic epithelium, such as expression of one or more genes selected from WT1 and GATA4. In some embodiments, the ovarian somatic cells can comprise or comprise characteristics of bipotential gonad, such as expression of one or more genes selected from WT1, GATA4, LHX9 and NR5A1. In some embodiments, somatic cells can comprise or comprise characteristics of Granulosa cells, such as expression of one or more genes selected from WT1, GATA4, LHX9, NR5A1 and FOXL2. In some embodiments, the ovarian somatic cells express one or more of the genes selected from WT1 and GATA4. In some embodiments, the ovarian somatic cells express WT1 and GATA4. In some embodiments, the ovarian somatic cells express one or more of the genes selected from WT1, GATA4, LHX9 and NR5A1. In some embodiments, the ovarian somatic cells express WT1, GATA4, LHX9 and NR5A1. In some embodiments, the ovarian somatic cells express one or more of the genes selected from WT1, GATA4, LHX9, NR5A1 and FOXL2. In some embodiments, the ovarian somatic cells express WT1, GATA4, LHX9, NR5A1 and FOXL2.

### C. Cell Mixture and Culture of Germ Cells and Somatic Cells

In some embodiments, germ cells and/or somatic cells are cultured together to form a cell mixture. Germ cells and/or somatic cells can be obtained by any suitable means, such as by any of the methods provided herein, e.g., in Section I.A. and Section I.B. In some embodiments a cell mixture comprising germ cells and somatic cells comprises germ cells that differentiate to oogonia and/or oocytes. In some embodiments, germ cells and/or somatic cells are cultured in a cell mixture to generate follicles.

In some embodiments, the germ and/or somatic cells are cultured together in a defined medium, such as any serum-free medium described herein, for example in Section II or the Examples, and comprising a low concentration of total protein or protein replacement (e.g., less than 3.5 mg/mL). In some of any of the provided embodiments, the defined medium comprises protein or protein replacement wherein the total concentration of the protein or protein replacement in the defined medium is lower than the concentration of protein or protein replacement in a standard culturing medium, such as comprising 10% fetal bovine serum (FBS). In some of any of the provided embodiments, the defined medium comprises protein or protein replacement wherein the total protein or protein replacement concentration in the defined medium is less than 3.5 mg/mL. In some embodiments, culture in the defined medium containing the low amount of protein or protein replacement increases survival, proliferation, and differentiation of germ cells (e.g., increases compared to culture in the same medium containing more than 3.5 mg/mL total protein or the same medium containing at least 10% fetal bovine serum).

In some embodiments of the provided methods, germ cells and somatic cells can be mixed in a cell mixture and cultured in the defined medium containing less than 3.5 mg/mL of a protein or protein replacement for a suitable amount of time, whereby the cells aggregate to form ovarian organoids.

In some embodiments, a cell mixture comprising germ cells and somatic cells are mixed for culture at a ratio of at or about 1:0.5, 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, or 1:20 germ cells to somatic cells, or any ratios in between. In some embodiments, the cell mixture comprising germ cells and somatic cells are mixed for culture at a ratio of at or about 1:2, 1:3, 1:4, 1:5, 1:6, 1:7, 1:8, 1:9, 1:10, 1:11, 1:12 of germ cells to somatic cells, or any ratios in between. In some embodiments, the cell mixture comprising germ cells and somatic cells are mixed for culture at a ratio of at or about 1:3 germ cells to somatic cells. In some embodiments, the cell mixture comprising germ cells and somatic cells are mixed for culture at a ratio of at or about 1:10 germ cells to somatic cells.

In some embodiments at least or at least about 5,000 of germ cells and somatic cells, combined, are mixed and cultured. In some embodiments, the total number of germ cells and somatic cells mixed to form the cell mixture is about 5,000 to about 250,000 total cells. In some embodiments, the total number of germ cells and somatic cells mixed to form the cell mixture is about 10,000 to about 225,000 total cells. In some embodiments, the total number germ cells and somatic cells mixed to form the cell mixture is about 15,000 to about 210,000 total cells. In some embodiments, the total number of cells germ cells and somatic cells mixed to form the cell mixture is about 20,000 to about 200,000 total cells.

In some embodiments, the cell mixture (i.e., germ cells and/or somatic cells) is cultured for a period of time sufficient to allow for aggregation. Following aggregation, the aggregated cell mixture of germ cells and somatic cells can form an ovarian organoid. In some embodiments, the period of time sufficient to allow for aggregation is about 12 to about 64 hours, or any time in between. In some embodiments, the period of time is about 18 to about 52 hours, or any time in between. In some embodiments, the period of time is about 24 hours to about 48 hours, or any time in between. In some embodiments, the cell mixture is cultured for about 24 hours to about 48 hours whereby the cells aggregate and form an ovarian organoid.

In some embodiments, the percentage of germ cells in the ovarian organoid is between at or about 0.1% and 90%, between 0.5% and 90%, between 1% and 90%, 0.1% and 80%, between 0.5% and 80%, between 1% and 80%, 0.1% and 70%, between 0.5% and 70%, between 1% and 70%, 0.1% and 60%, between 0.5% and 60%, between 1% and 60%, 0.1% and 50%, between 0.5% and 50%, between 1% and 50%, 0.1% and 40%, between 0.5% and 40%, between 1% and 40%, 0.1% and 30%, between 0.5% and 30%, between 1% and 30%, 0.1% and 20%, between 0.5% and 20%, between 1% and 20%, 0.1% and 10%, between 0.5% and 10%, or between 1% and 10% of the total number of cells in the organoid. In some embodiments, the percentage of germ cells in the ovarian organoid is between 1% and 20% or between 1% and 50% of the total number of cells in the organoid. In some embodiments, the percentage of germ cells in the ovarian organoid is between 1% and 20% of the total number of cells in the organoid. In some embodiments, the percentage of germ cells in the ovarian organoid is between 1% and 50% of the total number of cells in the organoid.

The culture of the germ cells and somatic cells into ovarian organoids can be by any suitable means, such as by any of the methods provided herein. In some embodiments, ovarian organoids can be cultured generally according to published methods of ovarian organoid culture, but in which the ovarian organoids are cultured in defined medium as described, such as a serum-free medium containing a low level of protein or protein replacement, such as less than 3.5 mg/mL total.

In some embodiments, ovarian organoids are kept or transferred to a suitable vessel for culturing. In some embodiments, the vessel is suitable for 3-Dimensional (3D) culture. Without being bound to a particular theory or mechanism, 3D culture can be effective for providing a scaffold for cell differentiation than two dimensional (2D) culture. Suitable 3D culture systems can include, for example, a hanging drop 3D culture, e.g., hanging drop plates, a 3D microwell culture, e.g., ultra-low attachment multiwell plates, a 3D culture on a hydrophobic surface, a rotational culture, a static 3D suspension culture, or a bioreactor. Hanging drop plates are commercially available such as, for example, the PERFECTA3D hanging drop plate, available from Biospherix, Parish, N.Y. Ultra-low attachment multiwell plates (in some cases also referred to as non-adherent culture vessels) are also commercially available such as, for example, AGGREWELL™ ultra-low attachment, multi-well plate, available from Stemcell Technologies, Vancouver, Canada.

In some embodiments, the vessel is not treated to promote cell adhesion and growth. In some embodiments, the vessel is a standard tissue culture plate that is not treated to promote cell adhesion and growth. In some embodiments, the cells do not adhere or substantially adhere during culture. In some embodiments, the cells are cultured in suspension.

In some embodiments, the vessels are multi-well plates. In some embodiments, the vessels are multi-well plates that are 96-well plates, 24-well plates or 6-well plates.

Organoids can be kept in an ultra low attachment plates whereby an organoid can float, wherein the organoid is in a fully immersed suspension culture. In some embodiments, the ovarian organoids are cultured in a fully immersed suspension culture. In some embodiments, the ovarian organoids are cultured in a fully immersed suspension culture indefinitely. In some embodiments, organoids can be transferred to air liquid interface mesh filters 48 hours after aggregation. In other embodiments, organoids can be transferred to an air liquid interface mesh filter after aggregation. In some embodiments, at or about 1-10 organoids per mesh filter are cultured in a tissue culture plate. In other embodiments, at or about 5-7 organoids per mesh filter are cultured in a tissue culture plate.

In some aspects, ovarian organoids can be assessed for characteristics such as cellular composition and morphology by any suitable means, such as any described herein and in the Examples. For example, ovarian organoids can be analyzed at regular time intervals, including analysis performed by next generation sequencing, histology, staining, and imaging.

In some embodiments, ovarian organoids are cultured in a defined medium, for example as described in Section II and in the Examples. In some embodiments, the defined medium does not comprise serum and comprises protein or protein replacement, and the concentration of the total protein or protein replacement in the defined medium is less than 3.5 mg/mL. In some embodiments, the ovarian orgaonids are cultured in the defined medium containing less than 3.5 mg/mL total protein or protein replacement until oocytes and/or oogonia are derived. In some embodiments, the ovarian organoids are further cultured until follicles are formed. In some embodiments, the further culturing is in the defined media or a different medium. In some embodiments, culture in the defined medium increases survival, proliferation, and differentiation of germ cells, to support generation of oocytes, oogonia, and/or follicles (e.g. primordial follicles) in the ovarian organoid culture.

### 1. Oogonia And/or Oocytes

In some embodiments, the germ cells are differentiated in culture into other germ cell types. For example, the germ cells can be, or can be differentiated into, oogonia and/or oocytes. In some embodiments, culturing the germ cells in the cell mixture described in Section I. C as part of the organoid culture, above induces differentiation of the germ cells into oogonia and/or ooctyes. In some embodiments, the culture of the germ cells and somatic cells as describes produces oogonia and/or oocytes.

For example, the germ cells can be, or can be differentiated into, oogonia. In some aspects, oogonia are germ cells that can differentiate into primary oocytes. In some aspects, oogonia express one or more genes selected from: DDX4, DAZL, STRA8, SYCP3, and SYCP1. In some embodiments, germ cells can be, or can be differentiated into, oocytes. In some embodiments, oocytes express one or more genes selected from FIGLA and ZP3.

In some embodiments, the germ cells in the cell mixture express one or more oogonia markers for meiotic entry, e.g., SYCP3 and SYCP1. In some embodiments, the germ cells express SYCP3 or SYCP1, and in some embodiments the germ cells express SYCP3 and SYCP1. In some embodiments, at least or at least about 1%, 2%, 3%, 4%, 5%, 6%, 7%, 8%, 9%, 10%, 11%, 12%, 13%, 14%, 15%, 16%, 18%, 19%, 20%, 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97%, or 98% of the germ cells express SYCP3 and/or SYCP1. In some embodiments, at least about 1% of the germ cells express SYCP3 and SYCP1. In some embodiments, at least about 5% of the germ cells express SYCP3 and SYCP1. In some embodiments, at least about 10% of the germ cells express SYCP3 and SYCP1. In some embodiments, at least about 20% of the germ cells express SYCP3 and SYCP1. In some embodiments, at least about 30% of the germ cells express SYCP3 and SYCP1. In some embodiments, at least about 40% of the germ cells express SYCP3 and SYCP1. In some embodiments, at least about 80% of the germ cells express SYCP3 and SYCP1. In some embodiments, at least about 90% of the germ cells express SYCP3 and SYCP1. In some embodiments, at least about 95% of the germ cells express SYCP3 and SYCP1.

In some embodiments, the germ cells in the cell mixture express DDX4. In some embodiments, the germ cells express DDX4. In some embodiments, at least about any one of: 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97%, or 98% of the germ cells express DDX4. In some embodiments, at least about 80% of the germ cells express DDX4. In some embodiments, at least about 90% of the germ cells express DDX4. In some embodiments, at least about 95% of the germ cells express DDX4.

In some embodiments, the germ cells in the cell mixture express DAZL. In some embodiments, the germ cells express DAZL. In some embodiments, at least about any one of: 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97%, or 98% of the germ cells express DAZL. In some embodiments, at least about 80% of the germ cells express DAZL. In some embodiments, at least about 90% of the germ cells express DAZL. In some embodiments, at least about 95% of the germ cells express DAZL.

As development progresses, oogonia begin to differentiate into oocytes, which are arrested in the first meiotic division. In some embodiments, oocytes express one or more genes selected from FIGLA, NOBOX, LMOD3 and ZP3.

In some embodiments, the oocytes in the cell mixture express one or more of genes FIGLA, NOBOX, LMOD3 and ZP3. In some embodiments, the oocytes express one or more of genes FIGLA, NOBOX, LMOD3 and ZP3. In some embodiments, at least about any one of: 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97%, or 98% of the oocytes express one or more genes FIGLA, NOBOX, LMOD3 and ZP3. In some embodiments, at least about 80% of the oocytes express one or more genes FIGLA, NOBOX, LMOD3 and ZP3. In some embodiments, at least about 90% of the oocytes express one or more of genes FIGLA, NOBOX, LMOD3 and ZP3. In some embodiments, at least about 95% of the oocytes express one or more genes FIGLA, NOBOX, LMOD3 and ZP3.

### 2. Primordial Follicles

In some embodiments, the cell mixture is cultured as part of the ovarian organoid is cultured until follicles are formed. In some embodiments, the method comprises further culturing the oogonia and/or oocytes to produce primordial follicles. In some embodiments the further culturing is performed in a medium that is the same or different from the defined medium.

In vivo, during the ovulation cycle, primordial follicles develop as primary follicles, secondary follicles, antral follicles, and Graffian follicles (mature follicles) to cause ovulation. In some embodiments, the provided methods allow for the generation of follicles, such as primordial follicles. In some embodiments, primordial follicles comprise an oocyte surrounded and/or contacted by a plurality of granulosa cells. In some embodiments herein, primordial follicles can be further activated, for example to produce primary follicles, secondary follicles, antral follicles, and/or Graffian follicles. In some embodiments, primordial follicles can be further activated, for example to produce primary follicles.

In some embodiments, the ovarian organoid develops into a primary follicle after a period of time in culture. In some embodiments, the period of time in culture for the development of primary follicle(s) is about 100 to about 160 days, or any time in between. In some embodiments, the period of time in culture for the development of primary follicles is at or about 100 days, 105, 110, 115, 120, 125, 130, 135, 140, 145, 150, 155, or 160 days, or any time in between. In some embodiments, the period of time in culture for the development of primary follicles is about 110 to about 150 days, or any time in between. In some embodiments, the period of time in culture for the development of primary follicles is about 120 to about 140 days, or any time in between. In some embodiments, the period of time in culture for the development of primary follicles is about 14 weeks, 15, 16, 17, 18, 19, 20, 21, 22, 23, or 24 weeks, or any time in between. In some embodiments, the period of time in culture for the development of primary follicles is 17, 28, 39, or 20 weeks, or any time in between. In some embodiments, the period of time in culture for the development of primary follicles is about 17 weeks to about 20 weeks. In some embodiments, the period of time in culture for the development of primary follicles is about 19 weeks. In some embodiments, the period of time in culture for the development of primary follicles is about 20 weeks.

In some embodiments, follicles are identified or characterized phenotypically. In some embodiments, follicles are identified by any suitable marker. In some embodiments, follicles are identified by an expressed gene or reporter. In some embodiments, follicles can be identified by any suitable marker(s) that identifies a germ cell (e.g., an oocyte) and a somatic cell. Markers for identifying follicles are known in the art and described herein. For example, germ cells (e.g., oogonia and/or ooctyes) can express DDX4 and somatic cells (e.g., granulosa cells) can express FOXL2, DDX4 and/or FOXL2 can be used as markers for particular cell types.

In some embodiments, follicles are DDX4+ and FOXL2+. In some embodiments, follicles comprise DDX4+ germ cells and FOXL2+ somatic cells. In some embodiments, follicles are characterized or identified by the presence of DDX4+ germ cells surrounded by FOXL2+ somatic cells. In some embodiments, follicles can be identified by morphological characteristics. In some embodiments, follicles exhibit a flattened morphology. In some embodiments, the follicles do not exhibit a flattened morphology. In some embodiments, developed follicles can remain in culture indefinitely.

### II. Defined Medium

In some embodiments, provided herein is a defined medium for use in connection with any of the methods provided herein, for example to produce oogonia, oocytes, and/or primordial follicles in ovarian organoid culture. The defined medium does not comprise serum, such as fetal bovine serum (FBS). In some embodiments, the provided methods are an improvement compared to published protocols for ovarian organoid culture. It is found herein that low protein in a defined medium, such as a a serum-free medium, is advantageous for promoting ovarian organoid culture.

In some embodiments, the defined medium is a serum free medium that contains protein or a protein replacement and has a total concentration of protein or protein replacement that is less than 3.5 mg/mL. In some embodiments, the defined medium containing a total concentration of protein or protein replacement that is less than 3.5 mg/mL supports improved survival, proliferation, and differentiation of germ cells in culture, for example in ovarian organoids. In some embodiments, the defined medium containing a total concentration of protein or protein replacement that is less than 3.5 mg/mL supports improved generation of follicles, such as primordial follicles. In some embodiments, the improvement is in comparison to the same culture conditions except that the culture medium contains FBS (e.g. 10% (v/v) FBS) in place of the protein or protein replacement. In some embodiments, the improvement is in comparison to culture conditions with media used in published protocols for ovarian organoid culture, such as media comprising FBS (e.g. 10% (v/v) FBS). In some embodiments, follicle generation in a defined medium comprising less than 3.5 mg/mL total protein or protein replacement is increased compared to follicle generation in a defined medium comprising greater than 3.5 mg/mL, such as, for example, at or about 4.8 mg/mL. In some embodiments, primordial follicle generation is increased by culture in a defined medium comprising at or about 3.5 mg/mL total protein or protein replacement compared to primordial follicle generation in a defined medium comprising greater than 3.5 mg/mL, such as, for example, at or about 4.8 mg/mL.

In some embodiments, the defined medium comprises one or more of any of the components described for the base medium or supplement medium described herein. In some embodiments the defined medium comprises a base media and a supplement media. In some examples, the defined medium comprises a base medium and a supplement media where the supplement media comprises a protein or protein replacement. In some examples, the defined medium comprises a base medium and a supplement media where the supplement media comprises a protein or protein replacement and wherein the concentration of total protein or protein replacement in the defined medium is less than 3.5 mg/mL. In some embodiments, the defined medium comprises the protein or protein replacement in a base medium. In some embodiments, the defined medium comprises the protein or protein replacement in a base medium, wherein the protein or protein replacement is a protein. In some embodiments, the defined medium comprises a protein or protein replacement in a base medium wherein the protein or protein replacement is a protein replacement. In some embodiments, the protein or protein replacement in the defined medium comprises serum protein components. In some embodiments, the protein or protein replacement in the defined medium comprises mammalian serum protein components. In some embodiments, the serum protein components are defined.

In some embodiments, the defined medium comprises a base medium and comprises a protein or a protein replacement. In some examples, the defined medium comprises a base medium and comprises a protein or protein replacement, such as any of the proteins or protein replacements provided herein, including any of the protein or protein replacements that can be included in Section II.B. In some embodiments the defined medium comprises a protein or protein substitute listed in Section II.B. In some embodiments, the defined medium comprises protein or protein replacement. In some embodiments, the total protein or protein replacement concentration in the defined medium is less than 3.5 milligrams per milliliter (mg/mL). In some embodiments, the protein or protein replacement is an albumin or albumin substitute. In some embodiments, the albumin is a human derived albumin. In some embodiments, the albumin is a recombinant albumin. In some embodiments, the albumin is a natural human serum albumin. In some embodiments, the albumin is a recombinant human serum albumin. In some embodiments, the albumin is a recombinant albumin from a non-human source. In some embodiments, the protein or protein replacement is or comprises a lipid enriched albumin. In some embodiments, the protein or protein replacement is or comprises a bovine serum albumin enriched with lipids. Albumin substitutes can be any protein or protein replacement source. Examples of such protein or protein replacement samples include but are not limited to bovine pituitary extract, plant hydrolysate (e.g., rice hydrolysate), fetal calf albumin (fetuin), egg albumin, human serum albumin (HSA), or another animal-derived albumins, chick extract, bovine embryo extract, AlbuMAX® I lipid rich bovine serum albumin, and AlbuMAX® II lipid rich bovine serum albumin. In some embodiments, the protein or protein replacement comprises a synthetic polymer. In some embodiments, the protein or protein replacement comprises polyvinyl alcohol (PVA) and/or Polyvinylpyrrolidone (PVP). In some embodiments, the protein replacement refers to any compound that can replace protein in the medium for use in the methods herein to give substantially similar results as protein. In some embodiments, a protein replacement functions like a protein and/or has the physical properties of the protein.

In some embodiments, the defined medium comprises a supplement medium. In some embodiments, the defined medium contains a defined serum replacement (DSR), optionally defined knockout serum replacement (KSR) and/or AlbuMAXII. In some embodiments, the defined medium contains 0-15% (v/v) defined knockout serum replacement (KSR), and/or 0-15% (v/v) AlbuMAXII. In some embodiments, the defined medium comprises a supplement medium that is a defined serum replacement, optionally defined knockout serum replacement (KSR).

In some embodiments, the defined medium is a defined, serum-free or serum-low media containing a dipeptide form of L-glutamine (e.g., L-alanyl-L-glutamine), a reducing agent (e.g., 2-mercaptoethanol), an antibiotic (e.g., penicillin-streptomycin), a vitamin (e.g., ascorbic acid) and at least one protein or protein replacement. In some embodiments, the defined medium comprises of GMEM (Gibco, Cat #:11710035), containing: 15% KnockOut™ Serum Replacement (Gibco, Cat #:10828028), 0.1 mM 2-ME (Fisher Chemical, Cat #:O3446I-100), 1x Glutamax™ dipeptide Supplement (Gibco, Cat #:35050061), 1x Pen/Strep (Gibco, Cat #:15070063), 0.1 mM NEAA (Gibco, Cat #:11140050), 1 mM Sodium Pyruvate (Gibco, Cat #:11360070), 10 μM Rock Inhibitor (Biogems, Cat #:1293823-10MG), and 100 μg/ml Primocin (Invivogen, Cat #:ant-pm-1).

In some embodiments, the defined medium is supplemented with at least one protein or protein replacement. In some embodiments, the defined medium comprises one or more of inorganic salts, sugars, amino acids, optionally also containing vitamins, organic acids, antioxidants, buffers; and the one protein or protein replacement. One or more further supplements can be added, including one or more supplements containing at least one protein, such as a serum-substituting protein, or one or more other components supporting growth and expansion of cells, such that the total protein or protein substitute in the medium is less than 3.5 mg/mL. In some embodiments, the at least one protein is a human protein or a recombinant protein. In some embodiments, the at least one protein is a human protein or a recombinant protein such as a serum-substituting protein, e.g. albumin. In some embodiments, the total protein and/or protein replacement concentration in the defined medium that contains protein and/or protein replacement is less than 3.5 mg/mL, less than 3.0 mg/mL, less than 2.5 mg/mL, less than 2.0 mg/mL, less than 1.5 mg/mL, less than 1.0 mg/mL, less than 0.5 mg/mL, or less than 0.1 mg/mL. In some embodiments, the total protein and/or protein replacement concentration in the defined medium is between about 0.1 mg/mL and 0.5 mg/mL, between about 0.5 mg/mL and 1.0 mg/mL, between about 1.0 mg/mL and 1.5 mg/mL, between about 1.5 mg/mL and 2.0 mg/mL, between about 2.0 mg/mL and 2.5 mg/mL, between about 2.5 mg/mL and 3.0 mg/mL, or between about 3.0 mg/mL and 3.5 mg/mL. In some embodiments, the total protein or protein replacement concentration in the defined medium is at or about 0.1%, 0.5%, 1%, 2%, 3%, 4%, 5%, 6%, 7%, 7.5% (v/v), or a value between any of the foregoing. In some embodiments, the total protein or protein replacement concentration in the defined medium is at or about 0.1 mg/mL, 0.2 mg/mL, 0.3 mg/mL, 0.4 mg/mL, 0.5 mg/mL, 0.6 mg/mL, 0.7 mg/mL, 0.8 mg/mL, 0.9 mg/mL, 1.0 mg/mL, 1.5 mg/mL, 2.0 mg/mL, 2.5 mg/mL, 3.0 mg/mL, or 3.5 mg/mL, or a value between any of the foregoing. In some embodiments, the total concentration of the protein or protein replacement is equal to or less than the concentration of protein from fetal bovine serum (FBS) in a medium comprising 7.5% (v/v) FBS. In some embodiments, the defined medium does not comprise serum. In some embodiments, the defined medium does not comprise FBS.

In some embodiments, the protein or protein replacement is comprised in a supplement medium that is present in the defined medium. The defined medium comprises a supplement medium comprising a protein or protein replacement. In some embodiments, the supplement medium comprises 7.5% (v/v) or less of the defined medium. In some embodiments, the supplement medium comprises between about 1% and 3%, between about 3% and 5%, or between about 5% and 7.5% (v/v) of the defined medium. In some embodiments, the supplement medium comprises at or about 0.5% (v/v), at or about 1.0% (v/v), at or about 1.5% (v/v), at or about 2.0% (v/v), at or about 2.5% (v/v), at or about 3.0% (v/v), at or about 3.5% (v/v), at or about 4.0% (v/v), at or about 4.5% (v/v), at or about 5.0% (v/v), at or about 5.5% (v/v), at or about 6.0% (v/v), at or about 6.5% (v/v), at or about 7.0% (v/v), or at or about 7.5% (v/v) of the defined medium, or a value between any of the foregoing. In some embodiments, the supplement medium comprises 2% (v/v) of the defined medium.

### A. Base Medium

In some embodiments, the defined medium can comprise a base medium, to which other components are added to obtain the defined medium. In embodiments herein, the base medium does not contain serum. The base medium can be any suitable base medium. The base medium can be a medium that is otherwise used for a wide variety of cell culture applications. In some embodiments, the base media contains non-essential amino acids. In some embodiments, the base medium comprises one or more amino acids. In some embodiments, the base medium comprises one or more of aspartic acid, glutamic acid, asparagine, serine, glutamine, histidine, glycine, threonine, arginine, alanine, tyrosine, cysteine, valine, methionine, norvaline, tryptophan, phenylalanine, isoleucine, leucine, lysine, hydroxyproline, sarcosine, and proline.

In some embodiments, the base medium is an artificial or synthetic medium. In some embodiments, the base medium is a balanced salt solution (e.g., PBS, DPBS, HBSS, EBSS). In some embodiments, the base medium is selected from Dulbecco's Modified Eagle's Medium (DMEM), Minimal Essential Medium (MEM), Basal Medium Eagle (BME), F-10, F-12, RPMI 1640, Glasgow's Minimal Essential Medium (GMEM), alpha Minimal Essential Medium (alpha MEM), advanced Minimal Essential Medium (advanced MEM), Iscove's Modified Dulbecco's Medium, and M199. In some embodiments, the base medium is a complex medium (e.g., RPMI-1640, IMDM). In some embodiments, the base medium is OpTmizer™ CTS™ T-Cell Expansion Basal Medium (ThermoFisher).

In some embodiments, the base medium comprises a nutrient mixture of inorganic salts, sugars, amino acids, optionally also containing vitamins, organic acids, antioxidants, and/or buffers.

In some embodiments, the base medium comprises CO₃ and HCO₃. In some embodiments, the content of CO₃/HCO₃ content of the base medium is balanced with gaseous CO₂ (e.g., 5-10%), thereby maintaining an optimal pH in the medium. In some embodiments, the base medium comprises a zwitterion, such as HEPES. In some embodiments, the base medium comprises phenol red. In some embodiments, the base medium does not comprise phenol red.

In some embodiments, the base medium comprises an inorganic salt. In some embodiments, the inorganic salt promotes the osmotic balance. In some embodiments, the inorganic salt regulates membrane potential by providing sodium, potassium, and calcium ions.

In some embodiments, the base medium comprises one or more carbohydrates. In some embodiments, the carbohydrate comprises glucose. In some embodiments, the carbohydrate comprises galactose. In some embodiments, the carbohydrate comprises maltose. In some embodiments, the carbohydrate comprises fructose.

In some embodiments, the base medium comprises fatty acid. In some embodiments, the base medium comprises lipid. In some embodiments, the base medium comprises vitamin (e.g., Vitamin A, Vitamin B7, Vitamin B9, Vitamin B12, Vitamin C, Vitamin E). In some embodiments, the base medium comprises a trace element. In some embodiments, the trace element comprises copper. In some embodiments, the trace element comprises zinc. In some embodiments, the trace element comprises selenium. In some embodiments, the trace element comprises tricarboxylic acid intermediate.

In some embodiments, the base medium contains a mixture of inorganic salts, sugars, amino acids, and, optionally, vitamins, organic acids and/or buffers or other well-known cell culture nutrients. In addition to providing nutrients, the base medium can also help maintain pH and osmolality. In some embodiments, the reagents of the base medium support cell growth, proliferation and/or expansion. A wide variety of commercially available base media are well known to those skilled in the art, and include Dulbeccos' Modified Eagles Medium (DMEM), Roswell Park Memorial Institute Medium (RPMI), Iscove modified Dulbeccos' medium and Hams medium. In some embodiments, the base medium is Iscove's Modified Dulbecco's Medium, RPMI-1640, advanced-MEM, or a-MEM.

In some embodiments, the base medium is free of a protein. In some embodiments, the base medium is free of a human protein (e.g., a human serum protein). In some embodiments, the base medium is serum-free. In some embodiments, the base medium is free of serum derived from fetal bovine.

In some embodiments, the base medium comprises Alpha MEM (Gibco, Cat #:12571063) or Advanced MEM (Gibco 12492013). In some embodiments, the base medium is further supplemented with: 1xGlutamax L-glutamine (Gibco, Cat #:35050061), 1x Pen/Strep (Gibco, Cat #:15070063), 50 μg/ml ascorbic acid (Sigma, Cat #:49752-10G), and 0.05-0.6 mM 2-ME (Fisher Chemical, Cat #:O3446I-100).

### B. Supplement Medium

In some embodiments, the protein or protein replacement is comprised in a supplement medium that is present in the defined medium. In some embodiments, the supplement medium is a defined serum replacement medium. In some embodiments, the supplement medium comprises 7.5% or less of the defined medium. In some embodiments, the supplement medium comprises between about 1% and 3%, between about 3% and 5%, or between about 5% and 7.5% of the defined medium. In some embodiments, the supplement medium comprises at or about 0.5%, at or about 1.0%, at or about 1.5%, at or about 2.0%, at or about 2.5%, at or about 3.0%, at or about 3.5%, at or about 4.0%, at or about 4.5%, at or about 5.0%, at or about 5.5%, at or about 6.0%, at or about 6.5%, at or about 7.0%, or at or about 7.5% of the defined medium, or a value between any of the foregoing. In some embodiments, the supplement medium comprises 2% of the defined medium. In some embodiments, the supplement such as these described herein is intended to be used as a media supplement (e.g., a media supplement for a base medium). In some embodiments, the supplement is intended to be used as a supplement for the maintenance, expansion, and/or activation of a cell. In some embodiments, the supplement is intended to be used as a supplement for the expansion of a cell.

In some embodiments, the supplement media comprises a protein or protein replacement, wherein the protein or protein replacement is an albumin or albumin substitute. In embodiments herein, a defined medium comprising a supplement medium contains a protein or protein replacement wherein the total protein or protein replacement is less than 3.5 mg/mL.

In some embodiments, the supplement medium is a defined serum replacement medium. In some embodiments, the supplement medium can be any suitable replacement medium, such as one described in WO 1998/030679A1, which is incorporated herein in its entirety.

In some embodiments, the supplement medium comprises at least one protein or protein replacement. In some embodiments, the at least one protein or protein replacement is at least one mammalian serum protein component. In some embodiments, the protein or protein replacement is from an animal source. In some embodiments, the protein or protein replacement is not fetal bovine serum. In some embodiments, the at least one protein includes albumin, transferrin, insulin, fibronectin, aprotinin or fetuin. In some embodiments, the protein or protein replacement comprises one or more of albumin, insulin, and transferrin, optionally one or more of a human or recombinant albumin, insulin, or transferrin.

In some embodiments, the protein or protein replacement in the supplement medium is an albumin or albumin substitute. In some embodiments, the protein in the supplement medium is an albumin that is a human derived albumin. In some embodiments, the albumin is a recombinant albumin. In some embodiments, the albumin is a natural human serum albumin. In some embodiments, the albumin is a recombinant human serum albumin. In some embodiments, the albumin is a recombinant albumin from a non-human source. Albumin substitutes can be any protein or protein replacement source. Examples of protein or protein replacement samples include but are not limited to bovine pituitary extract, plant hydrolysate (e.g., rice hydrolysate), fetal calf albumin (fetuin), egg albumin, human serum albumin (HSA), or another animal-derived albumins, chick extract, bovine embryo extract, AlbuMAX® I lipid rich bovine serum albumin, and AlbuMAX® II lipid rich bovine serum albumin. In some embodiments, the protein or protein replacement comprises a synthetic polymer. In some embodiments, the protein or protein replacement comprises polyvinyl alcohol (PVA) and/or Polyvinylpyrrolidone (PVP). In some embodiments, the protein replacement is a compound that can replace or substitute for protein in the medium for use in the methods described herein to give substantially similar results as protein. In some examples the protein replacement can act as a substrate for cell attachment. In some embodiments, the defined medium contains a defined serum replacement, optionally defined knockout serum replacement (KSR) and/or AlbuMAX® II lipid rich bovine serum albumin. In some embodiments, the defined medium contains 0-15 (v/v) % defined knockout serum replacement (KSR), and/or 0-15% (v/v) AlbuMAX® II lipid rich bovine serum albumin.

In some embodiments, the defined medium comprises a protein that is a transferrin or transferrin substitute. In embodiments herein, if the supplement media comprises a transferrin the supplement media also comprises another protein or protein replacement, wherein the total concentration of protein or protein replacement, including transferrin, is less than 3.5 mg/mL. In some embodiments, the transferrin or transferrin substitute is human transferrin or is derived from human transferrin. In some embodiments, the transferrin or transferrin substitute is derived from human serum or plasma. In some embodiments, the transferrin or transferrin substitute is recombinant transferrin. In some embodiments, the transferrin is iron saturated transferrin. In some embodiments, the transferrin is iron saturated human transferrin. In some embodiments, the concentration of the transferrin is such that after the supplement is combined with a base medium (such as these described herein), the concentration of the transferrin in the media is at or about 10 mg/L to at or about 50 mg/L, at or about 10 mg/L to at or about 100 mg/L, at or about 10 mg/L to at or about 150 mg/L, at or about 10 mg/L to at or about 200 mg/L, at or about 10 mg/L to at or about 250 mg/L, at or about 10 mg/L to at or about 300 mg/L, at or about 10 mg/L to at or about 350 mg/L, at or about 10 mg/L to at or about 400 mg/L, at or about 10 mg/L to at or about 450 mg/L, at or about 10 mg/L to at or about 500 mg/L, at or about 10 mg/L to at or about 550 mg/L, at or about 10 mg/L to at or about 600 mg/L, at or about 10 mg/L to at or about 650 mg/L, at or about 10 mg/L to at or about 750 mg/L, at or about 50 mg/L to at or about 100 mg/L, at or about 50 mg/L to at or about 150 mg/L, at or about 50 mg/L to at or about 200 mg/L, at or about 50 mg/L to at or about 250 mg/L, at or about 50 mg/L to at or about 300 mg/L, at or about 50 mg/L to at or about 350 mg/L, at or about 50 mg/L to at or about 400 mg/L, at or about 50 mg/L to at or about 450 mg/L, at or about 50 mg/L to at or about 500 mg/L, at or about 50 mg/L to at or about 550 mg/L, at or about 50 mg/L to at or about 600 mg/L, at or about 50 mg/L to at or about 650 mg/L, at or about 50 mg/L to at or about 750 mg/L, at or about 100 mg/L to at or about 150 mg/L, at or about 100 mg/L to at or about 200 mg/L, at or about 100 mg/L to at or about 250 mg/L, at or about 100 mg/L to at or about 300 mg/L, at or about 100 mg/L to at or about 350 mg/L, at or about 100 mg/L to at or about 400 mg/L, at or about 100 mg/L to at or about 450 mg/L, at or about 100 mg/L to at or about 500 mg/L, at or about 100 mg/L to at or about 550 mg/L, at or about 100 mg/L to at or about 600 mg/L, at or about 100 mg/L to at or about 650 mg/L, at or about 100 mg/L to at or about 750 mg/L, at or about 150 mg/L to at or about 200 mg/L, at or about 150 mg/L to at or about 250 mg/L, at or about 150 mg/L to at or about 300 mg/L, at or about 150 mg/L to at or about 350 mg/L, at or about 150 mg/L to at or about 400 mg/L, at or about 150 mg/L to at or about 450 mg/L, at or about 150 mg/L to at or about 500 mg/L, at or about 150 mg/L to at or about 550 mg/L, at or about 150 mg/L to at or about 600 mg/L, at or about 150 mg/L to at or about 650 mg/L, at or about 150 mg/L to at or about 750 mg/L, at or about 200 mg/L to at or about 250 mg/L, at or about 200 mg/L to at or about 300 mg/L, at or about 200 mg/L to at or about 350 mg/L, at or about 200 mg/L to at or about 400 mg/L, at or about 200 mg/L to at or about 450 mg/L, at or about 200 mg/L to at or about 500 mg/L, at or about 200 mg/L to at or about 550 mg/L, at or about 200 mg/L to at or about 600 mg/L, at or about 200 mg/L to at or about 650 mg/L, at or about 200 mg/L to at or about 750 mg/L, at or about 250 mg/L to at or about 300 mg/L, at or about 250 mg/L to at or about 350 mg/L, at or about 250 mg/L to at or about 400 mg/L, at or about 250 mg/L to at or about 450 mg/L, at or about 250 mg/L to at or about 500 mg/L, at or about 250 mg/L to at or about 550 mg/L, at or about 250 mg/L to at or about 600 mg/L, at or about 250 mg/L to at or about 650 mg/L, at or about 250 mg/L to at or about 750 mg/L, at or about 300 mg/L to at or about 350 mg/L, at or about 300 mg/L to at or about 400 mg/L, at or about 300 mg/L to at or about 450 mg/L, at or about 300 mg/L to at or about 500 mg/L, at or about 300 mg/L to at or about 550 mg/L, at or about 300 mg/L to at or about 600 mg/L, at or about 300 mg/L to at or about 650 mg/L, at or about 300 mg/L to at or about 750 mg/L, at or about 350 mg/L to at or about 400 mg/L, at or about 350 mg/L to at or about 450 mg/L, at or about 350 mg/L to at or about 500 mg/L, at or about 350 mg/L to at or about 550 mg/L, at or about 350 mg/L to at or about 600 mg/L, at or about 350 mg/L to at or about 650 mg/L, at or about 350 mg/L to at or about 750 mg/L, at or about 400 mg/L to at or about 450 mg/L, at or about 400 mg/L to at or about 500 mg/L, at or about 400 mg/L to at or about 550 mg/L, at or about 400 mg/L to at or about 600 mg/L, at or about 400 mg/L to at or about 650 mg/L, at or about 400 mg/L to at or about 750 mg/L, at or about 450 mg/L to at or about 500 mg/L, at or about 450 mg/L to at or about 550 mg/L, at or about 450 mg/L to at or about 600 mg/L, at or about 450 mg/L to at or about 650 mg/L, at or about 450 mg/L to at or about 750 mg/L, at or about 500 mg/L to at or about 550 mg/L, at or about 500 mg/L to at or about 600 mg/L, at or about 500 mg/L to at or about 650 mg/L, at or about 500 mg/L to at or about 750 mg/L, at or about 550 mg/L to at or about 600 mg/L, at or about 500 mg/L to at or about 650 mg/L, at or about 500 mg/L to at or about 750 mg/L, at or about 550 mg/L to at or about 600 mg/L, at or about 550 mg/L to at or about 650 mg/L, at or about 550 mg/L to at or about 750 mg/L, at or about 600 mg/L to at or about 650 mg/L, at or about 600 mg/L to at or about 750 mg/L, or at or about 650 mg/L to at or about 750 mg/L. In some embodiments, the concentration of the transferrin is such that after the supplement is combined with a basal medium (such as these described herein), the concentration of the transferrin in the media is at or about 100 mg/L. In some embodiments, the concentration of the transferrin is such that after the supplement is combined with a basal medium (such as these described herein), the concentration of the transferrin in the media is at or about 50 mg/L to at or about 150 mg/L. In embodiments herein, if the supplement media comprises a transferrin, the supplement media also comprises another protein or protein replacement, wherein the total concentration of protein or protein replacement, including a transferrin, is less than 3.5 mg/mL.

In some embodiments, the defined medium comprises a protein or protein replacement comprising an insulin or insulin replacement (substitute).

In embodiments herein, if the supplement media comprises an insulin, the supplement media also comprises another protein or protein replacement, wherein the total concentration of protein or protein replacement, including insulin, is less than 3.5 mg/mL. A number of insulins are known to those of ordinary skill in the art. See Gilman, A. G. et al, Eds., The Pharmacological Basis of Therapeutics, Pergamon Press, New York, 1990, pp. 1463-1495. Any known insulin can be included in the defined medium or supplement medium wherein the concentration of total protein in the defined medium, including the insulin, is less than 3.5 mg/mL. In some embodiments, insulin, rather than an insulin substitute, is used in the supplement medium and the defined medium. In some embodiments, the insulin is zinc insulin. In some embodiments, the insulin is human zinc insulin.

In some embodiments, the supplement media comprises an insulin that is a human insulin or derived from human insulin. In some embodiments, the supplement media comprises an insulin that is a recombinant insulin. In some embodiment, the insulin is a recombinant human insulin. In some embodiments, the concentration of the insulin (or insulin substitute) is such that after the supplement is combined with a basal medium (such as these described herein), at or about the concentration of the insulin (or insulin substitute) in the media is about 1 mg/L to at or about 2.5 mg/L, at or about 1 mg/L to at or about 5 mg/L, at or about 1 mg/L to at or about 7.5 mg/L, at or about 1 mg/L to at or about 10 mg/L, at or about 1 mg/L to at or about 12.5 mg/L, at or about 1 mg/L to at or about 15 mg/L, at or about 1 mg/L to at or about 17.5 mg/L, at or about 1 mg/L to at or about 20 mg/L, at or about 1 mg/L to at or about 22.5 mg/L, at or about 1 mg/L to at or about 25 mg/L, at or about 1 mg/L to at or about 27.5 mg/L, at or about 1 mg/L to at or about 30 mg/L, at or about 2.5 mg/L to at or about 5 mg/L, at or about 2.5 mg/L to at or about 7.5 mg/L, at or about 2.5 mg/L to at or about 10 mg/L, at or about 2.5 mg/L to at or about 12.5 mg/L, at or about 2.5 mg/L to at or about 15 mg/L, at or about 2.5 mg/L to at or about 17.5 mg/L, at or about 2.5 mg/L to at or about 20 mg/L, at or about 2.5 mg/L to at or about 22.5 mg/L, at or about 2.5 mg/L to at or about 25 mg/L, at or about 2.5 mg/L to at or about 27.5 mg/L, at or about 2.5 mg/L to at or about 30 mg/L, at or about 5 mg/L to at or about 7.5 mg/L, at or about 5 mg/L to at or about 10 mg/L, at or about 5 mg/L to at or about 12.5 mg/L, at or about 5 mg/L to at or about 15 mg/L, at or about 5 mg/L to at or about 17.5 mg/L, at or about 5 mg/L to at or about 20 mg/L, at or about 5 mg/L to at or about 22.5 mg/L, at or about 5 mg/L to at or about 25 mg/L, at or about 5 mg/L to at or about 27.5 mg/L, at or about 5 mg/L to at or about 30 mg/L, at or about 7.5 mg/L to at or about 10 mg/L, at or about 7.5 mg/L to at or about 12.5 mg/L, at or about 7.5 mg/L to at or about 15 mg/L, at or about 7.5 mg/L to at or about 17.5 mg/L, at or about 7.5 mg/L to at or about 20 mg/L, at or about 7.5 mg/L to at or about 22.5 mg/L, at or about 7.5 mg/L to at or about 25 mg/L, at or about 7.5 mg/L to at or about 27.5 mg/L, at or about 7.5 mg/L to at or about 30 mg/L, at or about 10 mg/L to at or about 12.5 mg/L, at or about 10 mg/L to at or about 15 mg/L, at or about 10 mg/L to at or about 17.5 mg/L, at or about 10 mg/L to at or about 20 mg/L, at or about 10 mg/L to at or about 22.5 mg/L, at or about 10 mg/L to at or about 25 mg/L, at or about 10 mg/L to at or about 27.5 mg/L, at or about 10 mg/L to at or about 30 mg/L, at or about 12.5 mg/L to at or about 15 mg/L, at or about 12.5 mg/L to at or about 17.5 mg/L, at or about 12.5 mg/L to at or about 20 mg/L, at or about 12.5 mg/L to at or about 22.5 mg/L, at or about 12.5 mg/L to at or about 25 mg/L, at or about 12.5 mg/L to at or about 27.5 mg/L, at or about 12.5 mg/L to at or about 30 mg/L, at or about 15 mg/L to at or about 17.5 mg/L, at or about 15 mg/L to at or about 20 mg/L, at or about 15 mg/L to at or about 22.5 mg/L, at or about 15 mg/L to at or about 25 mg/L, at or about 15 mg/L to at or about 27.5 mg/L, at or about 15 mg/L to at or about 30 mg/L, at or about 17.5 mg/L to at or about 20 mg/L, at or about 17.5 mg/L to at or about 22.5 mg/L, at or about 17.5 mg/L to at or about 25 mg/L, at or about 17.5 mg/L to at or about 27.5 mg/L, at or about 17.5 mg/L to at or about 30 mg/L, at or about 20 mg/L to at or about 22.5 mg/L, at or about 20 mg/L to at or about 25 mg/L, at or about 20 mg/L to at or about 27.5 mg/L, at or about 20 mg/L to at or about 30 mg/L, at or about 22.5 mg/L to at or about 25 mg/L, at or about 22.5 mg/L to at or about 27.5 mg/L, at or about 22.5 mg/L to at or about 30 mg/L, at or about 25 mg/L to at or about 27.5 mg/L, or at or about 27.5 mg/L to at or about 30 mg/L. In some embodiments, the concentration of insulin or insulin substitute in the media is at or about 10 mg/L. In some embodiments, the concentration of insulin or insulin substitute in the media is at or about 7.5 mg/L to at or about 12.5 mg/L.

In some embodiments, the supplement medium comprises a hormone (e.g., growth hormone, insulin, hydrocortisone, triiodothyronine, estrogen, androgen, progesterone, prolactin, follicle-stimulating hormone, gastrin-releasing peptide). In some embodiments, the supplement medium comprises alpha-globulin or beta-globulin.

In some embodiments, the supplement medium includes a peptide or peptide fraction (e.g., protein hydrolysate derived from animal, microorganism or plant).

In some embodiments, the total protein or protein replacement concentration in the defined medium is less than 3.5 mg/mL, less than 3.0 mg/mL, less than 2.5 mg/mL, less than 2.0 mg/mL, less than 1.5 mg/mL, less than 1.0 mg/mL, less than 0.5 mg/mL, or less than 0.1 mg/mL. In some embodiments, the total protein or protein replacement concentration in the defined medium is between about 0.1 mg/mL and 0.5 mg/mL, between about 0.5 mg/mL and 1.0 mg/mL, between about 1.0 mg/mL and 1.5 mg/mL, between about 1.5 mg/mL and 2.0 mg/mL, between about 2.0 mg/mL and 2.5 mg/mL, between about 2.5 mg/mL and 3.0 mg/mL, or between about 3.0 mg/mL and 3.5 mg/mL, each inclusive. In some embodiments, the total protein or protein replacement concentration in the defined medium is at or about 0.1%, 0.5%, 1%, 2%, 3%, 4%, 5%, 6%, 7%, 7.5%, each v/v, or a value between any of the foregoing. In some embodiments, the total protein or protein replacement concentration in the defined medium is at or about 0.1 mg/mL, 0.2 mg/mL, 0.3 mg/mL, 0.4 mg/mL, 0.5 mg/mL, 0.6 mg/mL, 0.7 mg/mL, 0.8 mg/mL, 0.9 mg/mL, 1.0 mg/mL, 1.5 mg/mL, 2.0 mg/mL, 2.5 mg/mL, 3.0 mg/mL, or 3.5 mg/mL, or a value between any of the foregoing.

In some embodiments, the supplement medium comprises one or more additional components. In some embodiments, the one or more additional components in the supplement includes a vitamin. In some embodiments, the vitamin comprises a fat-soluble vitamin (e.g., Vitamin A, Vitamin D, Vitamin E, Vitamin K). In some embodiments, the vitamin comprises a water-soluble vitamin (e.g., B1, B2, B6, B₁₂, C, folate).

In some embodiments, the one or more additional components includes transferrin substitute. In some embodiments, a transferrin substitute is a compound which can replace transferrin in the supplement to give substantially similar results as transferrin. Examples of transferrin substitutes include but are not limited to any iron chelate compound. Iron chelate compounds which can be used include but are not limited to iron chelates of ethylenediaminetetraacetic acid (EDTA), ethylene glycol-bis(β-aminoethyl ether)-N,N,N′,N′-tetraacetic acid (EGTA), deferoxamine mesylate, dimercaptopropanol, diethylenetriamine-pentaacetic acid (DTPA), and trans-1,2-diaminocyclohexane-N,N,N′,N′-tetraacetic acid (CDTA), as well as a ferric citrate chelate and a ferrous sulfate chelate.

In some embodiments, the one or more additional components in the supplement medium includes an insulin substitute that is not a protein. In some embodiments, the insulin substitute is a zinc containing compound which can be used in place of insulin to give substantially similar results as insulin. Examples of insulin substitutes include but are not limited to zinc chloride, zinc nitrate, zinc bromide, and zinc sulfate.

In some embodiments, the one or more additional components includes a lipid. In some embodiments, the lipid comprises cholesterol. In some embodiments, the lipid comprises steroid. In some embodiments, the lipid comprises fatty acid (e.g., palmitate, stearate, oleate, linoleate). In some embodiments, the lipid comprises ethanolamine. In some embodiments, the lipid comprises choline. In some embodiments, the lipid comprises inositol.

In some embodiments, the one or more additional components comprises a transition metal. In some embodiments, the transition metal comprises iron. In some embodiments, the transition metal comprises zinc. In some embodiments, the transition metal comprises copper. In some embodiments, the transition metal comprises chromium. In some embodiments, the transition metal comprises iodine. In some embodiments, the transition metal comprises cobalt. In some embodiments, the transition metal comprises selenium. In some embodiments, the transition metal comprises magnesium. In some embodiments, the transition metal comprises molybdenum. In some embodiments, the supplement medium can be any suitable replacement medium, such as KnockOut™ Serum Replacement. In some embodiments, the supplement medium comprises one or more ingredients selected from albumins or albumin substitutes, one or more amino acids, one or more vitamins, one or more transferrins or transferrin substitutes, one or more antioxidants, one or more insulins or insulin substitutes, one or more collagen precursors, and one or more trace elements. In some embodiments, the supplement medium is composed of albumins or albumin substitutes, one or more amino acids, one or more vitamins, one or more transferrins or transferrin substitutes, one or more antioxidants, one or more insulins or insulin substitutes, one or more collagen precursors, and one or more trace elements. In some embodiments, the supplement medium is composed of Transferrin (iron-saturated), insulin, lipid-rich albumin (AlbuMAX), one or more amino acids, one or more vitamins, one or more antioxidants, and one or more trace elements. In some embodiments, the supplement medium is composed of Transferrin (iron-saturated), insulin, lipid-rich albumin (AlbuMAX), Glycine, L-histidine, L-isoleucine, L-methionine, L-phenylalanine, L-proline, L-hydroxyproline, L-serine, L-threonine, L-tryptophan, L-tyrosine, L-valine, Thiamine, reduced glutathione, ascorbic acid 2-PO4, Ag+, Al3+, Ba2+, Cd2+, Co2+, Cr3+, Ge4+, Se4+, Br−, I−, F−, Mn2+, Si4+, V5+, Mo6+, Ni2+, Rb+, Sn2+, Zr4+.

Exemplary ingredients of a KnockOut™ Serum Replacement is set forth in Table 1

TABLE 1

| Ingredient | Examples |
|---|---|
| Amino Acids | Glycine, L-histidine, L-isoleucine, L-methionine, L- |
| | phenylalanine, L-proline, L-hydroxyproline, L-serine, |
| | L-threonine, L-tryptophan, L-tyrosine, L-valine |
| Vitamins/ | Thiamine, reduced glutathione, ascorbic acid 2-PO4 |
| Antioxidants | |
| Trace | Ag+, Al3+, Ba2+, Cd2+, Co2+, Cr3+, Ge4+, Se4+, |
| Elements | Br−, I−, F−, Mn2+, Si4+, V5+, Mo6+, Ni2+, Rb+, |
| | Sn2+, Zr4+ |
| Proteins | Transferrin (iron-saturated), insulin, lipid-rich |
| | albumin (AlbuMAX) |

### III. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The term “about” as used herein refers to the usual error range for the respective value readily known to the skilled person in this technical field. Reference to “about” a value or parameter herein includes (and describes) embodiments that are directed to that value or parameter per se.

As used herein, the singular forms “a,” “an,” and “the” include plural referents unless the context clearly dictates otherwise. For example, “a” or “an” means “at least one” or “one or more.”

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

As used herein, a composition refers to any mixture of two or more products, substances, or compounds, including cells. It may be a solution, a suspension, liquid, powder, a paste, aqueous, non-aqueous or any combination thereof.

As used herein, “optional” or “optionally” means that the subsequently described event or circumstance does or does not occur, and that the description includes instances where said event or circumstance occurs and instances where it does not. For example, an optionally substituted group means that the group is unsubstituted or is substituted.

The term “pharmaceutical composition” refers to a composition suitable for pharmaceutical use in a mammalian subject, often a human. A pharmaceutical composition typically comprises an effective amount of an active agent (e.g., cells, such as expanded in accord with the provided methods) and a carrier, excipient, or diluent. The carrier, excipient, or diluent is typically a pharmaceutically acceptable carrier, excipient or diluent, respectively.

A “pharmaceutically acceptable carrier” refers to a non-toxic solid, semisolid, or liquid filler, diluent, encapsulating material, formulation auxiliary, or carrier conventional in the art for use with a therapeutic agent that together comprise a “pharmaceutical composition” for administration to a subject. A pharmaceutically acceptable carrier is non-toxic to recipients at the dosages and concentrations employed and are compatible with other ingredients of the formulation. The pharmaceutically acceptable carrier is appropriate for the formulation employed.

The term “in vivo” refers to an event that takes plane in a mammalian subject's body.

The term “ex vivo” refers to an event that takes place on or in a tissue or cells from a mammalian subject but outside of the mammalian subject's body. Typically, the event is carried out in an external environment. In particular aspects, an ex vivo procedure includes any in which an organ, cell or tissue is taken from a subject, typically a living body, for a treatment or procedure and then returned to the subject.

The term “in vitro” refers to an event that takes place in a test system, such as in a laboratory.

As used herein, a kit is a packaged combination that optionally includes other elements, such as additional reagents and instructions for use of the combination or elements thereof.

The term “package insert” is used to refer to instructions customarily included in commercial packages of therapeutic products, that contain information about the indications, usage, dosage, administration, combination therapy, contraindications and/or warnings concerning the use of such therapeutic products.

As used herein, an “article of manufacture” is a product that is made and, in some cases, that can be sold. In some embodiments, the term can refer to compositions contained in articles of packaging, such as in a container.

It is understood that aspects and embodiments of the invention described herein include “comprising,” “consisting,” and “consisting essentially of” aspects and embodiments.

### IV. EXAMPLES

The following examples are included for illustrative purposes only and are not intended to limit the scope of the invention.

### Example 1: Generation of Organoid Culture and Analysis

Ovarian organoids containing in vitro-derived primordial germ cells (PGCs) and ovarian somatic cells were established for long-term culture and analysis.

Human in vitro-derived PGCs were derived from human pluripotent stem cells (hPSCs), expanded, dissociated, and sorted according to published protocols (e.g., as described in Irie et al., 2015 and Sasaki et al., Cell Stem Cell, 17(2):178-94, 2015). The hPSCs contained a constitutive fluorescent reporter for subsequent visualization of the in vitro-derived PGCs (PSC-derived fluorescent reporter). For FACS isolation, cultures were dissociated and PGCs were labeled with an anti-CD38 fluorescent antibody (Biolegend 303511) and sorted by FACS to obtain the isolated PGCs. Isolated PGCs were resuspended in aggregation media at a concentration of approximately 5,000 cells/100 μL media.

Aggregation medium was composed of GMEM (Gibco, Cat #:11710035), containing: 15% KnockOut™ Serum Replacement (Gibco, Cat #:10828028), 0.1 mM 2-ME (Fisher Chemical, Cat #:03446I-100), 1x Glutamax™ dipeptide Supplement (Gibco, Cat #:35050061), 1x Pen/Strep (Gibco, Cat #:15070063), 0.1 mM NEAA (Gibco, Cat #:11140050), 1 mM Sodium Pyruvate (Gibco, Cat #:11360070), 10 μM Rock Inhibitor (Biogems, Cat #:1293823-10MG), and 100 μg/ml Primocin (Invivogen, Cat #:ant-pm-1).

Fetal ovarian somatic cells were collected by mechanical dissection and dissociated to single cell suspension according to standard methods. Somatic cells were resuspended in aggregation medium at concentrations ranging from 45,000-180,000 cells/ 100 uL.

In vitro-derived PGCs and somatic cells were mixed together at a ratios of 1:10 to 1:3 PGCs: somatic cells, and plated in wells of a 96-well ultra-low attachment U bottom plate (Nexcelom, Cat #: ULA-96U), with 25,000-200,000 total cells per well. Cells were cultured fully submerged in aggregation suspension culture medium for 48 hours at 37 degrees, 5% CO2 to form cell aggregates (ovarian organoids or organoids). Cells were then transferred to long-term culture media in the same plates, or on Millicell Culture Plate Inserts (Thermo Fisher, Cat #:PICMO1250) immersed in long term culture media in a 24-well culture plate.

Long-term culture media was composed of either Alpha MEM (Gibco, Cat #:12571063) or Advanced MEM (Gibco 12492013), supplemented with: 1xGlutamax (Gibco, Cat #:35050061), 1x Pen/Strep (Gibco, Cat #:15070063), 50 μg/ml ascorbic acid (Sigma, Cat #:49752-10G), 0.05-0.6 mM 2-ME (Fisher Chemical, Cat #:O3446I-100), and varying amounts of fetal bovine serum (FBS) (Corning, Cat #: MT35015CV) or was formulated as a defined medium with a defined serum replacement (DSR), as described in the Examples below. In the Examples below, the defined medium is a media with varying concentration of defined serum replacement (DSR) (e.g., KnockOut Serum Replacement, Gibco, Cat. No. 10828028) in the AlphaMEM supplemented media. Media changes were performed every 2-3 days.

Ovarian organoids were collected for analysis at regular time intervals, including for next generation sequencing, histology, staining, and imaging, as described below and in the following Examples.

For live brightfield imaging, ovarian organoids were imaged throughout the culture lifetime using an EVOS M5000 (ThermoFisher) at 4× and 10× magnification.

For histological staining and analysis, organoids were collected, washed once in PBS and then fixed in 4% PFA (ThermoScientific J 61984.AP) for 30 min. Organoids were then washed once with PBS and stored in 70% Ethanol in sterile water solution. Organoids were processed on a Histocore Pegasus paraffin processor, and embedded in paraffin wax blocks.

Organoids were sectioned with 3-5 μm section thickness and stained using routine immuno-fluorescence practices (e.g. as described in Miller et al., Stem Cell Reports, 2018), and imaged by fluorescence microscopy.

### Example 2: Robust Survival and Proliferation of in Vitro-Derived Primordial Germ Cells in Ovarian Organoid Culture

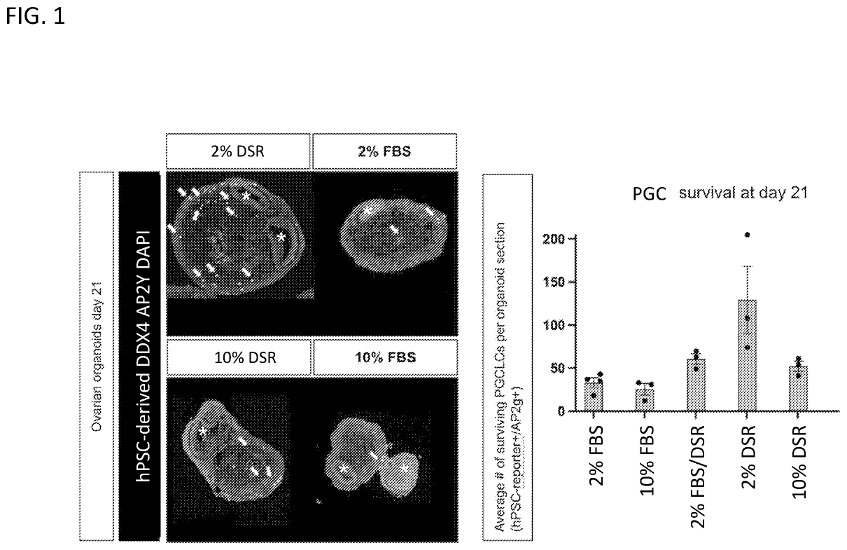

Ovarian organoids were prepared as described above in Example 1 and cultured in long-term media with 2% (v/v) FBS, 10% (v/v) FBS, or cultured in long-term media formulated as a defined medium containing 2% of the defined serum replacement (DSR), 10% DSR, or a mixture of the two long-term medias, 2% (v/v) FBS/DSR (a 1:1 mixture of 2% (v/v) FBS long-term media and 2% DSR long-term media). After 21 days, organoids were fixed, sectioned, and stained to quantify surviving PGCs per organoid. PGCs were derived from a fluorescently-tagged PSC reporter line, and were visualized by expression of the PSC-derived fluorescent reporter, and staining for AP2g, which marks PGCs. As shown in FIG. 1, conditions with long-term media that is defined medium containing 2% DSR resulted in dramatically increased survival of PGCs at day 21, in comparison to all other conditions.

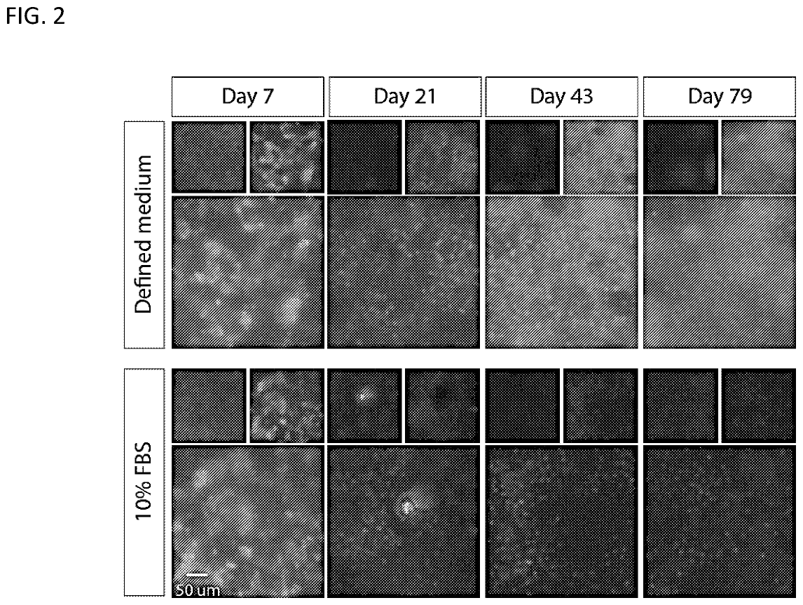

Ovarian organoids were prepared as described above in Example 1 and cultured in long-term media with 2% DSR (defined medium) or 10% (v/v) FBS. Organoids were analyzed in live culture by brightfield imaging (to visualize all cells) and fluorescent imaging (to visualize PSC-derived germ cells) at day 7, day 21, day 43, and day 79. As shown in FIG. 2, PSCs in 2% DSR conditions (defined medium) exhibited brighter fluorescence at day 21, day 43 and day 79 compared to conditions with 10% (v/v) FBS, the brighter fluorescence indicating robust survival and proliferation of the PSCs throughout the imaging timecourse. PSC survival and proliferation was dramatically improved in comparison to 10% (v/v) FBS conditions at day 21, day 43, and day 79.

The results support the utility of using a defined media with low protein concentration for ovarian organoid culture, including to support germ cell survival and proliferation.

### Example 3: Generation of Oocytes From in-Vitro Derived Primordial Germ Cells in Ovarian Organoid Culture

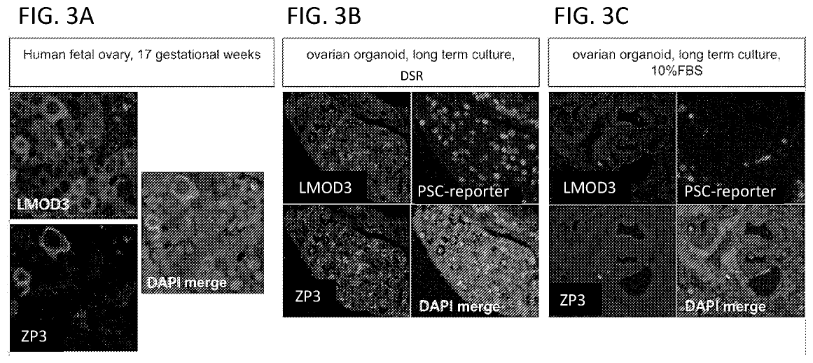

Ovarian organoids were prepared as described above in Example 1 and cultured in a long-term defined media with 2% defined serum replacement (DSR) or long-term media with 10% (v/v) FBS. After several weeks, organoids were fixed, sectioned, and stained to assess for the presence of germ cells competent for follicle assembly, as evidenced by expression of LMOD3 and ZP3 in germ cells expressing the fluorescent reporter. Human fetal ovary sections were also fixed and stained as a positive control. As shown in FIG. 3A, human fetal ovaries at 17 gestational weeks contained germ cells competent for follicle assembly, which co-expressed LMOD3 and ZP3. Shown in FIG. 3B, ovarian organoids cultured in long-term defined media with 2% DSR (defined medium) exhibited a high density of germ cells competent for follicle assembly, which expressed the PSC-derived fluorescent reporter, LMOD3, and ZP3. In contrast, in FIG. 3C, ovarian organoids cultured in long-term media with 10% (v/v) FBS exhibited few to no germ cells competent for follicle assembly, and had far fewer PSC-derived reporter-expressing germ cells overall.

The results support the utility of using a defined media with low protein concentration for ovarian organoid culture, including to support germ cell survival, proliferation, and differentiation to germ cells competent for follicle assembly.

### Example 4: Efficient Generation of Primordial Follicles in Ovarian Organoid Culture

Ovarian organoids were prepared as described above in Example 1 and cultured in long-term media with 2% DSR (defined medium) or long-term media with 10% (v/v) FBS.

After several weeks, organoids were fixed, sectioned, and stained to assess gene expression, cellular composition, and presence of primordial follicles in the organoids. Organoids were stained and imaged for the PSC-derived reporter (marking the germ cells), DDX4 (a marker of oogonia/oocytes expressed in the cytoplasm), and FOXL2 (a nuclear marker of ovarian somatic granulosa cells). Primordial follicles consist of a single DDX4+ oocyte surrounded by FOXL2+ somatic granulosa cells, which can exhibit a flattened morphology.

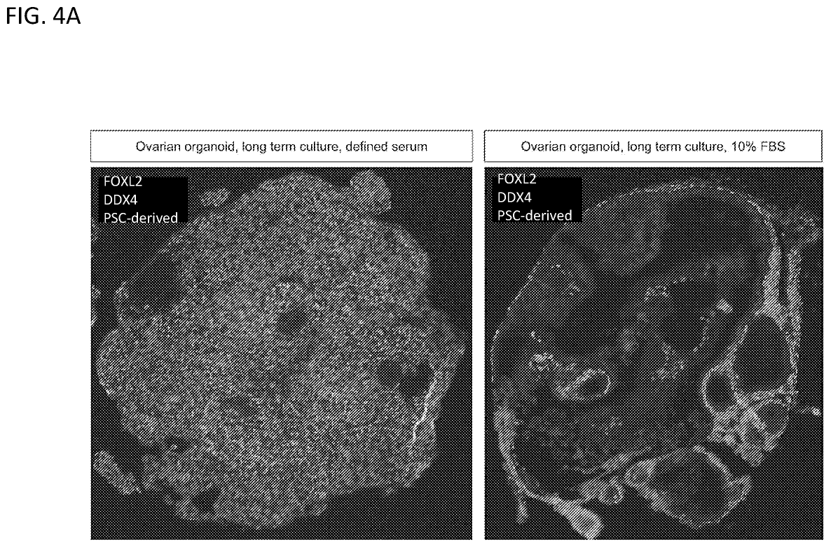

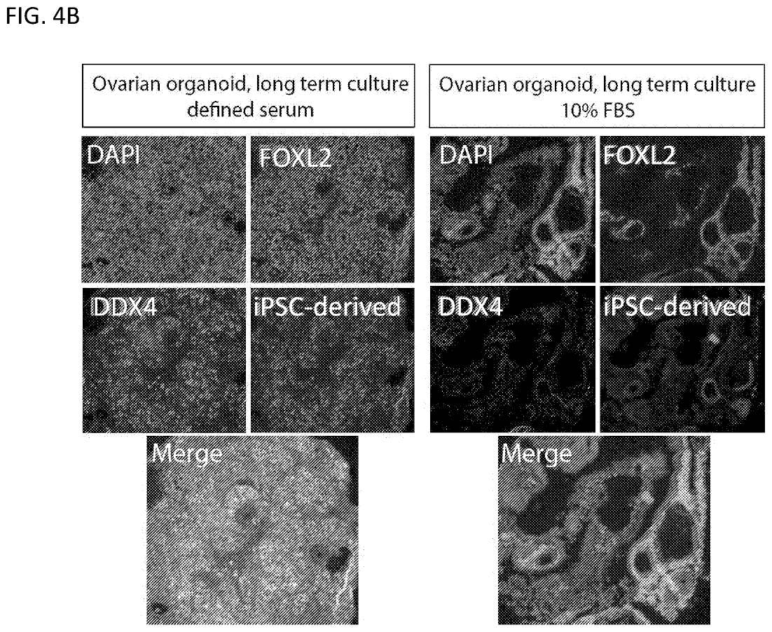

FIG. 4A and FIG. 4B show exemplary ovarian organoids from culture in long-term media with 2% DSR (defined medium) or long-term media with 10% (v/v) FBS. As shown in the figure, organoids cultured with 2% DSR exhibited robust expression of the PSC-derived reporter, DDX4, and FOXL2 scattered at a high density throughout the organoid, indicating robust survival, proliferation, and differentiation of the in vitro-derived PGCs into oogonia/oocytes, which were intermingled with somatic granulosa cells. In contrast, organoids cultured with 10% (v/v) FBS exhibited large areas without any cells, low survival of PSC-derived germ cells, and few FOXL2+ granulosa cells.

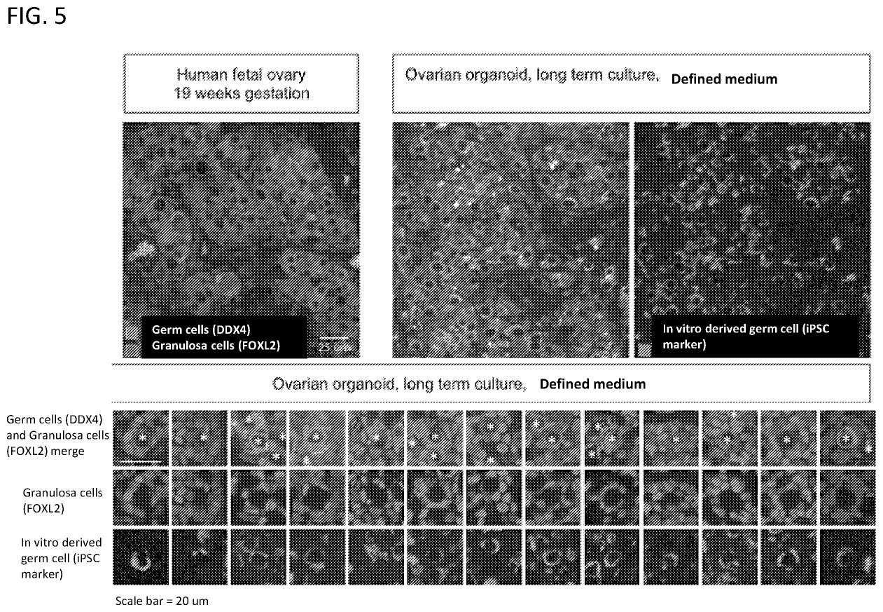

FIG. 5 shows a higher magnification image of an ovarian organoid cultured with 2% DSR (defined medium), which was stained and imaged as in FIG. 4, to assess for the presence of primordial follicles. Human fetal ovary sections were also fixed and stained as a positive control. Primordial follicles consist of a single DDX4+ oocyte surrounded by FOXL2+ somatic granulosa cells, which can exhibit a flattened morphology. As shown in FIG. 5, human fetal ovaries at 19 weeks gestation exhibited a high density of primordial follicles, characterized DDX4+ cells surrounded by FOXL2+ granulosa cells. Similarly, the ovarian organoids also exhibited a high density of primordial follicles, with PSC-derived reporter+/DDX4+ germ cells surrounded by FOXL2+ cells. Individual follicles with characteristic follicle morphology could be clearly visualized in the organoid, as shown in the several exemplary high magnification panels.

The results support the utility of using a defined media with low protein concentration for ovarian organoid culture, including to support germ cell survival, proliferation, differentiation, and follicle assembly.

### Example 5: Characterization of Intermediate Populations

Human in vitro-derived PGCLCs were derived from human pluripotent stem cells (hPSCs), expanded, dissociated, and sorted according to published protocols (e.g., as described in Irie et al., 2015 and Sasaki et al., Cell Stem Cell, 17(2):178-94, 2015), as described in Example 1. Ovarian organoids were prepared as described above in Example 1 and cultured in long-term defined media with 2% DSR (defined medium).

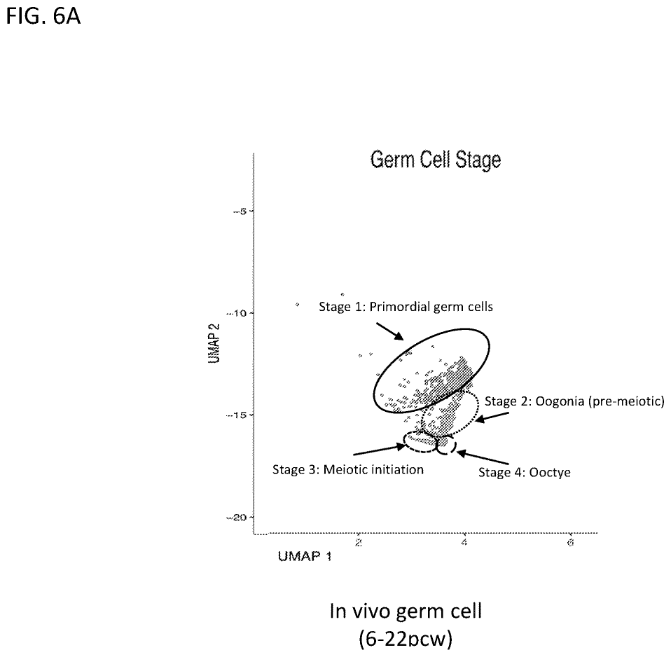

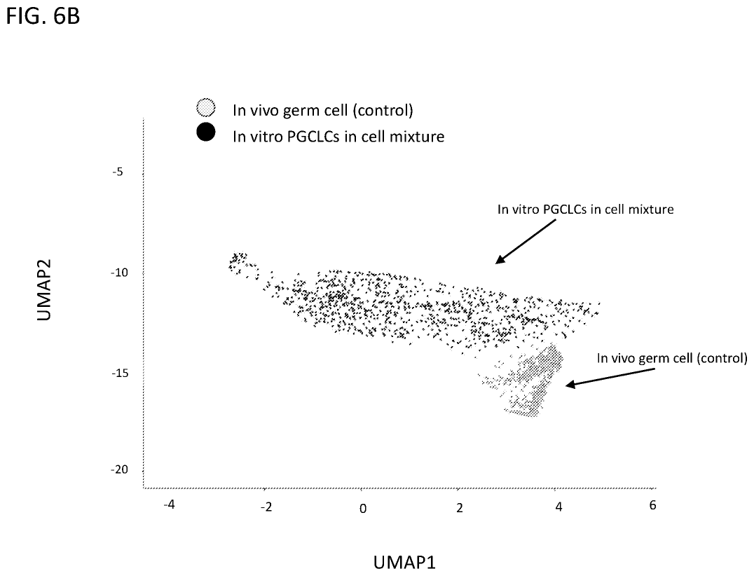

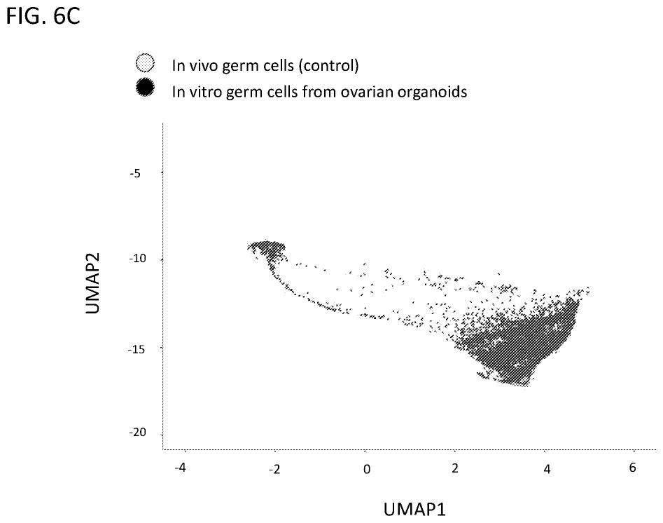

Single-cell RNA-sequencing of control human fetal germ cells from 6-22 post conception weeks (pwc), spanning stage 1 to stage 4 of development, was performed (FIG. 6A). Data were then merged with single-cell RNA-sequencing data from human pluripotent stem cell-derived germ cells from primordial germ cell like cell (PGCLC) starting cultures in the cell mixture (FIG. 6B) and after culture of the ovarian organoid (FIG. 6C). As seen in FIG. 6A FIG. 6C, after culture of the ovarian organoid in defined medium comtaining 2% defined serum replacement (e.g., 2% KSR), a subset of pluripotent stem cell-derived in vitro cultured human germ cells overlaped directly with human fetal germ cells spanning from stage 1 to stage 4, indicating in vitro cultured germ cells progress in this culture system.

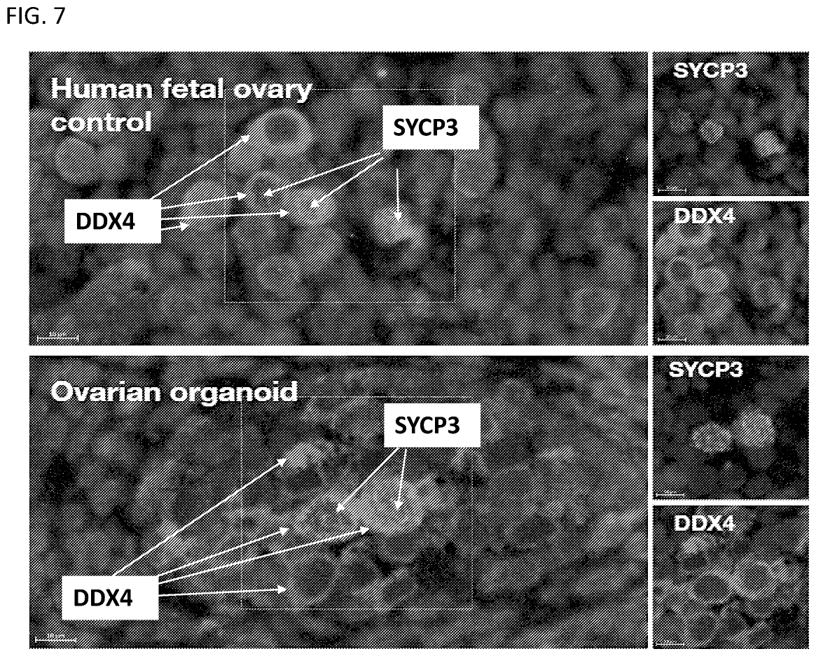

FIG. 7 shows a cultured ovarian organoid (bottom panels) prepared according to the method described in Example 1, above, compared to a human fetal ovary control (top panels). As shown in the figure, the germ cells (identified by positive DDX4 staining) in both the human fetal ovary control and the ovarian organoid showed positive expression of nuclear SYP3+ staining, indicating that the germ cells have undergone meiotic entry. The results show that human pluripotent stem cell-derived germ cells cultured in accord with the described methods can progress to meiotic entry in ovarian organoids cultures.

The present invention is not intended to be limited in scope to the particular disclosed embodiments, which are provided, for example, to illustrate various aspects of the invention. Various modifications to the compositions and methods described will become apparent from the description and teachings herein. Such variations can be practiced without departing from the true scope and spirit of the disclosure and are intended to fall within the scope of the present disclosure.

1. A method of producing primordial follicles, the method comprising:
a) providing a cell mixture of germ cells and somatic cells in culture, and
b) culturing the cell mixture for a period of time in a defined medium to produce the primordial follicles,
wherein the defined medium comprises protein or protein replacement, and the total protein or protein replacement concentration in the defined medium is less than 3.5 milligrams per milliliter (mg/mL).

2. The method of claim 1, wherein each primordial follicle comprises (i) an oocyte, and (ii) a plurality of granulosa cells, optionally wherein the plurality of granulosa cells are the somatic cells in a) or are derived from the somatic cells in a).

3. A method of producing oogonia and/or oocytes, the method comprising:
a) providing a cell mixture of germ cells and somatic cells in culture, and
b) culturing the cell mixture for a period of time in a defined medium to produce the oogonia and/or ooctyes,
wherein the defined medium comprises protein or protein replacement, and the total protein or protein replacement concentration in the defined medium is less than 3.5 milligrams per milliliter (mg/mL).

4. The method of claim 3, wherein the method comprises further culturing the oogonia and/or oocytes to produce primordial follicles.

5. The method of claim 4, wherein the further culturing is performed in a medium that is the same or different from the defined medium.

6. The method of any of claims 1-5, wherein the total protein or protein replacement concentration in the defined medium is less than 3.5 mg/mL, less than 3.0 mg/mL, less than 2.5 mg/mL, less than 2.0 mg/mL, less than 1.5 mg/mL, less than 1.0 mg/mL, less than 0.5 mg/mL, or less than 0.1 mg/mL.

7. The method of any of claims 1-6, wherein the total protein or protein replacement concentration in the defined medium is between about 0.1 mg/mL and 0.5 mg/mL, between about 0.5 mg/mL and 1.0 mg/mL, between about 1.0 mg/mL and 1.5 mg/mL, between about 1.5 mg/mL and 2.0 mg/mL, between about 2.0 mg/mL and 2.5 mg/mL, between about 2.5 mg/mL and 3.0 mg/mL, or between about 3.0 mg/mL and 3.5 mg/mL.

8. The method of any of claims 1-7, wherein the total protein or protein replacement concentration in the defined medium is at or about 0.1 mg/mL, 0.2 mg/mL, 0.3 mg/mL, 0.4 mg/mL, 0.5 mg/mL, 0.6 mg/mL, 0.7 mg/mL, 0.8 mg/mL, 0.9 mg/mL, 1.0 mg/mL, 1.5 mg/mL, 2.0 mg/mL, 2.5 mg/mL, 3.0 mg/mL, or 3.5 mg/mL, or a value between any of the foregoing.

9. The method of any of claims 1-8, wherein the defined medium is a serum-free medium that does not comprise serum.

10. The method of any of claims 1-9, wherein the defined medium does not comprise FBS.

11. The method of any of claims 1-10, wherein the defined medium comprises:
one or more of: inorganic salt(s), sugar(s), amino acid(s), vitamin(s), organic acid(s), antioxidant(s), and buffers; and
the protein or protein replacement.

12. The method of any of claims 1-11, wherein the defined medium comprises the protein or protein replacement in a base medium.

13. The method of claim 12, wherein the base medium comprises one or more of Dulbecco's Modified Eagle's Medium (DMEM), Minimal Essential Medium (MEM), Basal Medium Eagle (BME), F-10, F-12, RPMI 1640, Glasgow's Minimal Essential Medium (GMEM), alpha Minimal Essential Medium (alpha MEM), advanced Minimal Essential Medium (advanced MEM), Iscove's Modified Dulbecco's Medium, and M199.

14. The method of any of claims 1-13, wherein the protein or protein replacement is a protein.

15. The method of any of claims 1-14, wherein the protein or protein replacement is a protein replacement.

16. The method of any of claims 1-15, wherein the protein or protein replacement is or comprises albumin or an albumin substitute.

17. The method of any of claims 1-16, wherein the protein or protein replacement comprises one or more components selected from: bovine pituitary extract, plant hydrolysate (e.g., rice hydrolysate), an albumin, chick extract, bovine embryo extract, fetal calf albumin (fetuin), egg albumin, human serum albumin (HSA), other animal-derived albumins, and a bovine serum albumin.

18. The method of any of claims 1-17, wherein the protein or protein replacement is or comprises a lipid enriched albumin.

19. The method of any of claims 1-18, wherein the protein or protein replacement is or comprises a bovine serum albumin enriched with lipids.

20. The method of any of claims 1-19, wherein the protein or protein replacement is or comprises AlbuMAX® lipid rich bovine serum albumin, optionally AlbuMAX® I lipid rich bovine serum albumin or AlbuMAX® II lipid rich bovine serum albumin.

21. The method of any of claims 1-20, wherein the defined medium comprises a synthetic component.

22. The method of any of claims 1-21, wherein the protein or protein replacement comprises a synthetic polymer.

23. The method of claim 22, wherein the synthetic polymer is polyvinyl alcohol (PVA) and/or polyvinylpyrrolidone (PVP).

24. The method of any of claims 12-23, wherein the defined medium is prepared by adding a protein or protein replacement to the base medium.

25. The method of any of claims 12-24, wherein the protein or protein replacement is provided by a supplement medium that is added to the base medium.

26. The method of claim 25, wherein the supplement medium is a defined serum replacement medium.

27. The method of 25 or 26 wherein the supplement medium is added to the base medium to a final concentration of 7.5% or less.

28. The method of any of claims 25-27, wherein the supplement medium is added to the base medium to a final concentration (v/v) of between about 1% and 7.5%, between about 1% and 5%, between about 1% and 3%, between about 3% and 5%, or between about 5% and 7.5%.

29. The method of any of claims 25-28, wherein the supplement medium is added to the base medium to a final concentration of at or about 0.5%, at or about 1.0%, at or about 1.5%, at or about 2.0%, at or about 2.5%, at or about 3.0%, at or about 3.5%, at or about 4.0%, at or about 4.5%, at or about 5.0%, at or about 5.5%, at or about 6.0%, at or about 6.5%, at or about 7.0%, or at or about 7.5%, or a value between any of the foregoing.

30. The method of any of claims 25-27, wherein the supplement medium is added to the base medium to a final concentration of 2%.

31. The method of any of claims 25-30, wherein the supplement medium comprises one or more ingredients selected from the group consisting of albumins or albumin substitutes, one or more amino acids, one or more vitamins, one or more transferrins or transferrin substitutes, one or more antioxidants, one or more insulins or insulin substitutes, one or more collagen precursors, and one or more trace elements.

32. The method of any of claims 25-31, wherein the supplement medium comprises an albumin or albumin substitute.

33. The method of any of claims 11-32, wherein the antioxidant is selected from the group consisting of reduced glutathione, ascorbic acid, and ascorbic acid-2-phosphate.

34. The method of any of claims 31-33, wherein the collagen precursor is selected from the group consisting of L-proline and multimers or derivatives thereof, L-hydroxyproline multimers or derivatives thereof, and ascorbic acid or multimers thereof.

35. The method of any of claims 31-34, wherein the transferrin substitute is an iron chelate selected from the group consisting of a ferric citrate chelate and a ferrous sulfate chelate, optionally ferrous sulphate-7 water-EDTA.

36. The method of any of claims 31-35, wherein the insulin substitute is selected from the group consisting of zinc chloride, zinc bromide, and zinc sulfate-7 water.

37. The method of any of claims 31-36, wherein the amino acid ingredient comprises one or more amino acids selected from the group consisting of glycine, L-alanine, L-asparagine, L-cysteine, L-aspartic acid, L-glutamic acid, L-phenylalanine, L-histidine, L-isoleucine, L-lysine, L-leucine, L-glutamine, L-arginine, L-methionine, L-proline, L-hydroxyproline, L-serine, L-threonine, L-tryptophan, L-tyrosine, and L-valine, and derivatives thereof.

38. The method of any of claims 31-37, wherein the trace element ingredient comprises one or more trace element moieties selected from the group consisting of Ag⁺, Al³⁺, Ba²⁺, Cd²⁺, Co²⁺, Cr³⁺, Ge⁴⁺, Se⁴⁺, Br⁻, I⁻, Mn²⁺, F⁻, Si⁴⁺, V⁵⁺, Mo⁶⁺, Ni²⁺, Rb⁺, Sn²⁺, and Zr⁴⁺.

39. The method of any of claims 25-38, wherein the supplement medium comprises lipid-rich albumin (AlbuMAX), L-glycine, L-histidine, L-isoleucine, L-methionine, L-phenylalanine, L-proline, L-hydroxyproline, L-serine, L-threonine, L-tryptophan, L-tyrosine, L-valine, thiamine, reduced glutathione, L-ascorbic acid-2-phosphate, iron saturated transferrin, insulin, sodium selenite, Ag⁺, Al³⁺, Ba²⁺, Cd²⁺, Co²⁺, Cr³⁺, Ge⁴⁺, Se⁴⁺, Br⁻, I⁻, Mn²⁺, F⁻, Si⁴⁺, V⁵⁺, Mo⁶⁺, Ni²⁺, Rb⁺, Sn²⁺, and Zr⁴⁺.

40. The method of any of claims 25-39, wherein the supplement medium is knockout serum replacement (KSR).

41. The method of any of claims 1-40, wherein the defined medium is a base medium supplemented with 1-5% defined knockout serum replacement (KSR).

42. The method of any of claims 1-41, wherein the cell mixture is an ovarian organoid.

43. The method of claim 42, wherein the ovarian organoid is cultured in a fully immersed suspension culture.

44. The method of claim 42 or 43, wherein the ovarian organoid is cultured at an air-liquid interface using permeable culture membranes.

45. The method of any of claims 1-44, wherein the method further comprises removing the defined medium from the cell mixture after oogonia and/or oocytes are produced.

46. The method of any of claims 42-45, wherein the ovarian organoid is composed of 500-300,000 cells.

47. The method of any of claims 42-46, wherein the percentage of germ cells in the ovarian organoid is between 1% and 20%, or between 1% and 50%, of the total number of cells in the organoid.

48. The method of any of claims 1-47, wherein the cell mixture is cultured for between 1 day and 300 days in vitro.

49. The method of any of claims 1-48, wherein the period of time to produce the primordial follicles is between about 100 days and about 160 days, between about 110 days and about 150 days, or between about 120 and about 140 days in culture.

50. The method of any of claims 1-49, wherein the period of time to produce the primordial follicles is between about 110 and about 150 days in culture.

51. The method of any of claims 1-50, wherein the period of time to produce the primordial follicles is between about 120 and about 140 days in culture.

52. The method of any of claims 1, 2, and 4-51, wherein the method further comprises activating the primordial follicles to produce primary follicles.

53. The method of any one of claims 1, 2, and 4-52, wherein the method further comprises activating the primordial follicles to produce primary follicles, secondary follicles, antral follicles, and/or Graffian follicles.

54. The method of any of claims 1-53, wherein the germ cells are primordial germ cells (PGCs).

55. The method of any of claims 1-53, wherein the germ cells are primordial germ cell-like cells PGCLCs.

56. The method of claim 54 or 55, wherein at least or at least about 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97%, or 98% of the germ cells express one or more primordial germ cell marker genes, optionally selected from: NANOS3, SOX17, NANOG, TBXT, TFAP2C, PRDM1, and POU5F1.

57. The method of claim 54 or 55, wherein the germ cells express one or more primordial germ cell marker genes, optionally selected from: NANOS3, SOX17, NANOG, TBXT, TFAP2C, PRDM1, and POU5F1.

58. The method of any of claims 54-57, wherein the germ cells express one or more primordial germ cell marker genes, optionally selected from: TFAP2C, PRDM1, and POU5F1.

59. The method of any of claims 54-58, wherein at least or at least about 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97%, or 98% of the germ cells do not express one or more alternative lineage markers.

60. The method of any of claims 54-59, wherein the germ cells do not express one or more alternative lineage markers.

61. The method of any of claims 54-60, wherein the germ cells do not express one or more alternative lineage markers, optionally selected from: FOXA2, HHEX, CDX2, and SOX2.

62. The method of any of claims 1-61, wherein the germ cells are mammalian, optionally human, non-human primate, pig, cow, or horse.

63. The method of any of claims 1-62, wherein the germ cells are derived from in vivo tissue.

64. The method of any of claims 1-63, wherein the germ cells are derived from stem cells, optionally pluripotent stem cells (PSCs), optionally induced pluripotent stem cells (iPSCs) or embryonic stem cells (ESCs).

65. The method of any of claims 1-64, wherein the germ cells differentiate into the oogonia and/or oocytes.

66. The method of any of claims 3-65, wherein the oogonia express one or more oogonia marker genes, optionally selected from: DDX4, DAZL, STRA8, and ZGLP1, and/or wherein the germ cells express one or more oogonia markers for meiotic entry, optionally selected from SYCP3 and SYCP1.

67. The method of any of claims 3-65, wherein at least or at least about 1%, 2%, 3%, 4%, 5%, 6%, 7%, 8%, 9%, 10%, 11%, 12%, 13%, 14%, 15%, 16%, 18%, 19%, 20%, 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97%, or 98% of the oogonia express one or more oogonia marker genes, optionally selected from: DDX4, DAZL, STRA8, and ZGLP1, and/or wherein at least or at least about 1%, 2%, 3%, 4%, 5%, 6%, 7%, 8%, 9%, 10%, 11%, 12%, 13%, 14%, 15%, 16%, 18%, 19%, 20%, 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97%, or 98% of the germ cells express one or more oogonia markers for meiotic entry, optionally selected from SYCP3 and SYCP1.

68. The method of any of claims 3-65, wherein at least or at least about 1% of the oogonia express one or more oogonia marker genes, optionally selected from: DDX4, DAZL, STRA 8, and ZGLP1, and/or wherein at least or at least about 1% of the germ cells express one or more oogonia markers for meiotic entry, optionally selected from SYCP3 and SYCP1.

69. The method of any of claims 3-65, wherein at least or at least about 5% of the oogonia express one or more oogonia marker genes, optionally selected from: DDX4, DAZL, STRA 8, and ZGLP1, and/or wherein at least or at least about 5% of the germ cells express one or more oogonia markers for meiotic entry, optionally selected from SYCP3 and SYCP1.

70. The method of any of claims 2-69, wherein the oocytes express one or more oocyte marker genes, optionally selected from FIGLA, ZP3, and LMOD3.

71. The method of any of claims 2-70, wherein at least or at least about 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97%, or 98% of the oocytes express one or more oocyte marker genes, optionally selected from FIGLA, ZP3, and LMOD3.

72. The method of any of claims 2-71, wherein at least or at least about 60% of the oocytes express one or more oocyte marker genes, optionally selected from FIGLA, ZP3, and LMOD3.

73. The method of any of claims 2-72, wherein at least or at least about 70% of the oocytes express one or more oocyte marker genes, optionally selected from FIGLA, ZP3, and LMOD3.

74. The method of any of claims 2-73, wherein at least or at least about 80% of the oocytes express one or more oocyte marker genes, optionally selected from FIGLA, ZP3, and LMOD3.

75. The method of any of claims 2-74, wherein at least or at least about 90% of the oocytes express one or more oocyte marker genes, optionally selected from FIGLA, ZP3, and LMOD3.

76. The method of any of claims 1-75, wherein the somatic cells are mammalian ovarian somatic cells.

77. The method of any of claims 1-76, wherein the somatic cells are primary cells from a mammal.

78. The method of any of claims 1-77, wherein the somatic cells are primary cells from a fetal mammalian ovary.

79. The method of any of claims 1-78, wherein the somatic cells are from human, non-human primate, pig, rabbit, cow, mouse, rat, donkey, and/or rabbit.

80. The method of any of claims 1-79, wherein the somatic cells are differentiated in vitro from another cell type.

81. The method of any of claims 1-80, wherein the somatic cells are differentiated from stem cells, optionally pluripotent stem cells (PSCs), optionally induced pluripotent stem cells (iPSCs) or embryonic stem cells (ESCs).

82. The method of any of claims 1-81, wherein the somatic cells comprise one or more ovarian somatic cell types.

83. The method of any of claims 1-82, wherein one or more of the somatic cells types expresses one or more genes characteristic of intermediate mesoderm, including WT1.

84. The method of any of claims 1-83, wherein one or more of the somatic cells types expresses one or more genes characteristic of coelomic epithelium, including WT1 and GATA4.

85. The method of any of claims 1-84, wherein one or more of the somatic cells types expresses one or more genes characteristic of granulosa cells, including WT1, GATA4, LHX9, NR5A1, and/or FOXL2.

86. The method of any of claims 1-85, wherein one or more of the somatic cell types expresses one or more genes characteristic of bipotential gonad, including WT1, GATA4, LHX9, and/or NR5A1.

87. A composition comprising primordial follicles produced by the method of any of claims 1-86.

88. An ovarian organoid produced by the method of any of claims 46-74.

89. An ovarian organoid comprising one or more primordial follicles, wherein: each primordial follicle comprises (i) an oocyte differentiated in culture from mammalian pluripotent stem cells, and (ii) a plurality of granulosa cells.

90. The ovarian organoid of claim 89, wherein the pluripotent stem cells are from human, non-human primate, pig, cow, or horse.

91. The ovarian organoid of claim 89 or 90, wherein the ovarian organoid comprises at least 5 primordial follicles, at least 10 primordial follicles, at least 20 primordial follicles, at least 50 primordial follicles, at least 100 primordial follicles, at least 500 primordial follicles, or more that 500 primordial follicles.

92. An ovarian organoid, wherein the ovarian organoid is derived from a cell mixture of ovarian germ cells and somatic cells in culture, and the ovarian organoid comprises at least 5, at least 10, at least 50, at least 100, or at least 500 oocytes and/or oogonia cells derived from the ovarian germ cells, wherein the ovarian germ cells are from human or non-human primate.

93. The ovarian organoid of claim 92, wherein the ovarian germ cells are human germ cells.

94. The ovarian organoid of claim 92 or 93, wherein the ovarian germ cells are primary germ cells (PGCs) or primary germ cell-like cells (PGCLCs).

95. The ovarian organoid of claim 94, wherein the primary germ cells (PGCs) or primary germ cell-like cells (PGCLCs) undergo meiotic entry.

96. The ovarian organoid of any of claims 92-95, wherein the ovarian germ cells differentiate in the ovarian organoid, wherein the differentiated ovarian germ cells are phenotypically similar to human fetal germ cells from in vivo tissue.

97. The ovarian organoid of any of claims 92-96, wherein the differentiated ovarian germ cells express one or more primordial germ markers, one or more oogonia markers, one or more markers for meiotic initiation, and/or one or more oocyte markers.

98. The ovarian organoid of any of claims 92-97, wherein the differentiated ovarian germ cells express markers of the germ cell stages of human fetal germ cells from in vivo tissue.

99. The ovarian organoid of any of claims 92-98, wherein the ovarian germ cells differentiate in the ovarian organoid, wherein the differentiated ovarian germ cells are phenotypically similar to the germ cell stages of human fetal germ cells from in vivo tissue.

100. The ovarian organoid of claim 99, wherein the germ cell stages progress from primordial germ cells, oogonia, meiotic initiation, and oocyte.

101. The ovarian organoid of any of claims 92-100, wherein the percentage of oocytes, oogonia, and/or other germ cells in the ovarian organoid is more than 1%, more than 5%, more than 10%, more than 20%, more than 30%, more than 40%, or more than 50% of the total number of cells in the ovarian organoid.

102. The ovarian organoid of any of claims 92-101, wherein the ovarian germ cells of the cell mixture exhibit at least 1%, at least 5%, at least 10%, at least 20%, at least 50%, or greater survival after at least 1 week in culture.
